(19) United States
(12) Patent Application Publication
(10) Pub. No.: US 2015/0125550 A1
(43) Pub. Date: May 7, 2015

(54) APPLICATION OF 2,9-DI-SEC-BUTYL-1,10-PHENANTHROLINE AS A GLIOBLASTOMA TUMOR CHEMOTHERAPY

(71) Applicants: Jack F. Eichler, Riverside, CA (US); Clement N. David, Palm Springs, CA (US); Emma H. Wilson, Riverside, CA (US)

(72) Inventors: Jack F. Eichler, Riverside, CA (US); Clement N. David, Palm Springs, CA (US); Emma H. Wilson, Riverside, CA (US)

(21) Appl. No.: 14/473,539

(22) Filed: Aug. 29, 2014

Related U.S. Application Data
(60) Provisional application No. 61/871,852, filed on Aug. 29, 2013.

(51) Int. Cl. A61K 31/555; A61K 45/06; A61N 5/10; A61K 31/4745

(52) U.S. Cl. CPC A61K 31/555; A61K 31/4745; A61K 45/06; A61N 5/10
USPC 424/649; 435/375; 514/292; 514/185; 546/88; 546/10; 600/1

(57) ABSTRACT

A method of inhibiting cancer cell growth is provided. In some versions, the method includes exposing lung cancer cells or glioma cells to 2,9-di-sec-butyl-1,10-phenanthroline (SBP) or derivatives of SBP in an amount effective to inhibit glioma cell growth. Also, a method of treating a lung cancer or a glioma tumor in a subject in need of such treatment is provided. The method includes administering SBP or derivatives of SBP to the subject in an amount effective to treat the lung cancer or glioma tumor. For either method, the method can further include exposing or administering pseudo five coordinate gold(III) complexes of SBP derivatives.

### BACKGROUND

1. Field of the Invention

The invention relates to the treatment of glioblastoma tumors.

2. Related Art

Glioblastoma multiforme is an extremely aggressive and invasive form of central nervous system (CNS) tumor associated with a prognosis of less than one year survival. Current therapies employ surgical removal in combination with radiation therapy and chemotherapy. Though this removes a large part of the tumor, it often does not eliminate all tumor cells, and relapses occur quickly. Furthermore, chemotherapy and radiation therapy crudely target large regions of the body and can leave the patients with substantial deleterious side effects. Temozolomide (TMZ) is a chemotherapeutic drug in use since 1999 to treat advanced glioblastomas and melanomas. Its anti-tumor effects stem from its capability in methylating DNA at the N-7 or O-6 position of guanine, damaging the DNA and thereby causing cell death. Unfortunately, some patients relapse with TMZ-resistant disease. The resistance to current chemotherapies, limited success of treatment, and poor long term prognosis warrants the search for and creation of new drugs, which alone or in combination with other forms of therapy could target and eradicate tumor cells more efficiently.

Cisplatin has been used to treat humans afflicted with lung cancer in a clinical setting. Cisplatin has been observed to improve patient survival, particularly for patients with early stage lung cancer or advanced disease with good prognosis. However patients treated with cisplatin experience several unwanted side effects (including nausea, myelosuppression, neurotoxicity, and renal function impairment) (see the following literature citation for more detailed analysis of lung cancer studies with cisplatin: Journal of the National Cancer Institute, 2007, 99 (11), 847-857, incorporated by reference herein). Therefore alternative treatments are continually being investigated.

### SUMMARY

In one aspect, a method of inhibiting glioma cell growth is provided. The method includes exposing glioma cells to at least one antitumor compound in an amount effective to inhibit growth of the glioma cells, where the antitumor compound is selected from the group consisting of: 2,9-di-sec-butyl-1,10-phenanthroline (SBP); (2,9-di-sec-butyl-1,10-phenanthroline)AuCl₃; (2-mono-n-butyl-phenanthroline)AuCl₃; and a combination thereof.

In the method: a) the glioma cells can be glioblastoma cells; b) the method can further include exposing the glioma cells to an anticancer agent; c) the anticancer agent can be a platinum-based compound; d) the platinum-based compound can be cisplatin; e) or any combination of a)-d).

In another aspect, a method of treating a glioma tumor in a subject in need of such treatment is provided. The method includes administering at least one antitumor compound to the subject in an amount effective to treat the tumor, where the antitumor compound is selected from the group consisting of: 2,9-di-sec-butyl-1,10-phenanthroline; (2,9-di-sec-butyl-1,10-phenanthroline)AuCl₃; (2-mono-n-butyl-phenanthroline)AuCl₃; and a combination thereof.

In the method: a) the glioma tumor can be a glioblastoma; b) the method can further include administering an anticancer agent to the subject; c) the anticancer agent can be a platinum-based compound; d) the platinum-based compound can be cisplatin; e) the method can further include administering an anticancer treatment to the subject; f) the anticancer treatment can be surgery, chemotherapy, radiotherapy or immunotherapy; g) or any combination of a)-f).

In a further aspect, a method of inhibiting cancer cell growth is provided. The method includes exposing cancer cells to at least one antitumor compound in an amount effective to inhibit growth of the cancer cells. In the method, the cancer cells can be lung or glioma cancer cells, and the antitumor compound is selected from the group consisting of: 2,9-di-n-butyl-1,10-phenanthroline; 2,9-di-sec-butyl-4-methyl-1,10-phenanthroline; 2-sec-butyl-1,10-phenanthroline; 2-mono-n-butyl-phenanthroline; 2,9-di-phenyl-1,10-phenanthroline; 2,9-di-phenyl-4-methyl-1,10-phenanthroline; 2-mono-sec-butyl-2,2′-bipyridine; (2,9-di-n-butyl-1,10-phenanthroline)AuCl₃; (2,9-di-methyl-1,10-phenanthroline)AuCl₃; (2,9-di-sec-butyl-4-methyl-1,10-phenanthroline)AuCl₃; (2-sec-butyl-1,10-phenanthroline)AuCl₃; (2-mono-n-butyl-phenanthroline)AuCl₃; (2,9-di-phenyl-1,10-phenanthroline)AuCl₃; (2,9-di-phenyl-4-methtyl-1,10-phenanthroline)AuCl₃; 2-mono-sec-butyl-2,2′-bipyridine)AuCl₃; and a combination thereof.

In the method: a) the cancer cells can be lung cancer cells; b) the cancer cells can be glioma cells; c) the compound can be (2,9-di-n-butyl-1,10-phenanthroline)AuCl₃; d) the compound can be (2-mono-n-butyl-phenanthroline)AuCl₃; e) or any combination of a)-d).

Also in the method, when the cancer cells are glioma cells, the compound can be 2,9-di-methyl-1,10-phenanthroline.

In another aspect, a method of treating cancer in a subject in need of such treatment is provided. The method includes administering at least one antitumor compound to the subject in an amount effective to treat the cancer. In the method, the cancer can be lung cancer or glioma cancer, and the antitumor compound is selected from the group consisting of: 2,9-di-n-butyl-1,10-phenanthroline; 2,9-di-sec-butyl-4-methyl-1,10-phenanthroline; 2-sec-butyl-1,10-phenanthroline; 2-mono-n-butyl-phenanthroline; 2,9-di-phenyl-1,10-phenanthroline; 2,9-di-phenyl-4-methyl-1,10-phenanthroline; 2-mono-sec-butyl-2,2′-bipyridine; (2,9-di-n-butyl-1,10-phenanthroline)AuCl₃; (2,9-di-methyl-1,10-phenanthroline)AuCl₃; (2,9-di-sec-butyl-4-methyl-1,10-phenanthroline)AuCl₃; (2-sec-butyl-1,10-phenanthroline)AuCl₃; (2-mono-n-butyl-phenanthroline)AuCl₃; (2,9-di-phenyl-1,10-phenanthroline)AuCl₃; (2,9-di-phenyl-4-methtyl-1,10-phenanthroline)AuCl₃; 2-mono-sec-butyl-2,2′-bipyridine)AuCl₃; and a combination thereof.

In the method: a) the cancer can be lung cancer; b) the cancer can be glioma cancer or tumor; c) the compound can be (2,9-di-n-butyl-1,10-phenanthroline)AuCl₃; d) the compound can be (2-mono-n-butyl-phenanthroline)AuCl₃; e) the method can further include administering an anticancer agent to the subject; f) the anticancer agent can be a platinum-based compound; g) the platinum-based compound can be cisplatin; h) the method can further include administering an anticancer treatment to the subject; i) the anticancer treatment can be surgery, chemotherapy, radiotherapy or immunotherapy; j) or any combination of a)-i).

Also in the method, when the cancer is a glioma cancer or tumor, the compound can be 2,9-di-methyl-1,10-phenanthroline.

In another aspect, a compound is provided. The compound is selected from the group consisting of: 2,9-di-sec-butyl-4-methyl-1,10-phenanthroline; 2-sec-butyl-1,10-phenanthroline; 2-mono-n-butyl-phenanthroline; 2,9-di-phenyl-1,10-phenanthroline; 2,9-di-phenyl-4-methyl-1,10-phenanthroline; 2-mono-sec-butyl-2,2′-bipyridine; (2,9-di-sec-butyl-4-methyl-1,10-phenanthroline)AuCl₃; (2-sec-butyl-1,10-phenanthroline)AuCl₃; (2-mono-n-butyl-phenanthroline)AuCl₃; (2,9-di-phenyl-1,10-phenanthroline)AuCl₃; (2,9-di-phenyl-4-methtyl-1,10-phenanthroline)AuCl₃; and (2-mono-sec-butyl-2,2′-bipyridine)AuCl₃. Further, a pharmaceutical composition is provided comprising one or a combination of these compounds, and a pharmaceutically acceptable carrier.

### BRIEF DESCRIPTION OF THE DRAWINGS

For a more complete understanding of the present invention, reference is now made to the following descriptions taken in conjunction with the accompanying drawings, in which:

FIG. 1 is a panel showing in-vitro GL-26 inhibition with SBP. Chemical structure of MSBP and SBP (1A). GL-26 cells were grown in a 96 well plate and treated with SBP, MSBP or TMZ at 0.1 to 25 uM (Student's T-test, MSBP: p<0.0001 and TMZ: p=0.0005 compared to SBP) (1B). To test SBP toxicity on non-tumor cells, primary murine astrocytes and human foreskin fibroblasts (HFFs) were plated and treated as above (Student's T-test, astrocytes: p=0.0001 and HFFs: p=0.0002 compared to GL-26) (C). The Sulforhodamine B colorimetric assay (SRB) was used to measure and plot fraction of growth of treated wells compared to a non-treated control.

FIG. 2 is a panel showing that compound SBP induces apoptosis. GL-26 cells were grown in a 96 well plate and treated with 0.4 to 25 uM SBP. After 48 hr incubation, the drug was removed and cells allowed to recover for another 48 hrs in fresh media. Sulforhodamine B colorimetric assay (SRB) was used to measure and plot fraction of growth of treated wells compared to a non-treated control (2A). Propidium iodide staining intensity was measured by flow cytometry and plotted vs cell number to identify cell cycle stages (2B). S: Synthesis, M: Mitotic. SBP treated and untreated cultured GL-26 cells were stained for apoptosis (TUNEL). Positive control was treated with the kit's nuclease to generate DNA breaks in every cell (2C).

FIG. 3 is a panel to show that SBP inhibits in-vivo glioma growth. Tumor bearing mice were treated on day 1, 7 and 13 post injection and sacrificed on day 19. Mouse weights were recorded during the 19-day trial. A best-fit line (not shown) reveals a significant weight decrease in not-treated animals (p=0.0261) but not in SBP treated animals (p=0.8792) (3A). Hematoxylin and eosin stained ex-vivo coronal slices were taken from SBP treated mice and non-treated mice (N=3 for each group) (3B) and tumor section area quantified (Student's T-test, not-treated: 48187±7736, SBP treated: 5489±1369 p=0.0056). T=tumor (3C). Ex-vivo slices were stained for apoptosis (TUNEL) in SBP treated (middle panel) and nontreated mice (right panel, 3D). Positive control was treated with the kit's nuclease to generate DNA breaks in every cell (left panel, 3D).

FIG. 4 is a panel to show that SBP does not cause peripheral pathology: Liver, lung and gut 6 μm thick section from SBP treated and non-treated were collected, stained with hematoxylin and eosin and assessed blindly by a trained pathologist (4A). Blood samples from the same cohort were tested for levels of alanine aminotransferase and aspartate aminotransferase (Student's T-test, ALT: p=0.2596 and AST p=0.3982) (4B).

FIG. 5 is a panel of graphs of SRB data for cisplatin/SBP combination therapies. FIGS. 5A and 5B depict fraction of cell growth for specific concentrations of cisplatin/SBP. FIG. 5C depicts the entire growth curve from 0.1-25 μM for the individual SBP (labeled “SBP”, cisplatin (labeled “cis”), and SBP/cisplatin combination therapies (labeled “0.1’ or “0.4”, respectively).

FIG. 6 is a panel of X-ray crystal structures for various compounds. In FIGS. 6A-6E, atoms shown in gray are carbon, atoms shown in white are hydrogen (these atom labels are omitted for sake of clarity).

FIG. 7 is a panel of graphs of representative GSH stability profiles shown for: (FIG. 7A) [(^{di-methyl}phen)AuCl₃] (12); (FIG. 7B) [(^{mono-sec-butyl}phen)AuCl₃] (14), and (FIG. 7C) [(^{di-sec-butyl-methyl}phen)AuCl₃] (13). 5.0×10⁻⁵ M solutions of the gold complexes were prepared in phosphate buffer (0.10M, pH 7.4) possessing one mole equivalent of reduced glutathione (GSH). UV-visible spectra were collected once every hour over a 15 hour period.

FIG. 8 is a panel of graphs showing the activity of gold complexes against GL-26 murine glioma cells. FIG. 8A shows activity for SBP, and FIGS. 8B-8C show activity for gold complexes.

### DETAILED DESCRIPTION

The following application is incorporated by reference herein: U.S. Provisional Patent Application No. 61/871,852, filed on Aug. 29, 2013.

A potential alternative chemotherapy to the drug TMZ has been identified as the compound 2,9-di-sec-butyl-1,10-phenanthroline (SBP), which has been observed to have potent in vitro and in vivo antitumor activity against the highly aggressive gliomablastoma cell line GL-26. In vitro testing clearly indicates that SBP has significantly higher activity than the currently used therapy, TMZ, as SBP has IC₅₀ values against GL-26 tumor cells that are approximately 3-6 μM, whereas TMZ shows no inhibition of tumor cell growth up to 25 μM. In vitro tests also indicate that SBP is significantly less toxic to non-cancerous human fibroblast cells and non-cancerous glioma cells (IC₅₀ is not reached on either of these normal cell lines at 25 μM). In vitro tests also indicate that SBP appears to kill GL-26 tumor cells, and does not appear to simply inhibit cell growth.

In vivo testing of SBP has also been carried out on a mouse model, where GL-26 brain tumors were implanted in mice. This in vivo study indicates that SBP significantly reduces brain tumor growth, as tumors in treated mice were 5-10 times smaller than tumors in untreated control mice. Additionally, healthy mice that were treated with SBP did not appear to experience any significant side effects, as tissues in these mice were found to be similar to healthy mice not treated with the drug.

In embodiments of the present invention, compounds may be formulated as pharmaceutical compositions. Pharmaceutical compositions of the present invention comprise, for example, SBP and a pharmaceutically acceptable carrier and/or diluent. Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990. The compound may thus be administered in dosage formulations containing conventional non-toxic pharmaceutically acceptable carriers, adjuvants and vehicles.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions may, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan mono-laurate, triethanolamine acetate, triethanolamine oleate, etc. Actual methods of preparing such dosage forms are known, or will be apparent, to those skilled in this art. For oral administration, the composition will generally take the form of a tablet or capsule, or may be an aqueous or nonaqueous solution, suspension or syrup. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

In some embodiments, exposure to or treatment with SBP can be combined with other anti-cancer treatments, such as surgery, chemotherapy, radiotherapy or immunotherapy. For example, exposure to or treatment with SBP can be combined with exposure to or treatment with TMZ and/or radiotherapy.

For exposure or treatment, routes of administration will vary, naturally, with the location and nature of the lesion, and include, e.g., intratumoral, intracranial, intravenous, intradermal, transdermal, parenteral, intramuscular, intranasal, subcutaneous, percutaneous, intratracheal, intraperitoneal, perfusion, lavage, direct injection, and oral administration.

In embodiments where a glioma tumor, lung cancer, or other cancer or tumor in a subject is treated, an amount effective to treat the cancer or tumor is an amount that promotes or enhances the well-being, of the subject with respect to the medical treatment of the subject's condition. A list of nonexhaustive examples of this includes extension of the subject's life by any period of time, a decrease in pain to the subject that can be attributed to the subject's condition, a decrease in the severity of the disease, an increase in the therapeutic effect of a therapeutic agent, au improvement in the prognosis of the condition or disease, a decrease in the amount or frequency of administration of a therapeutic agent, an alteration in the treatment regimen of the subject that reduces invasiveness of treatment, a decrease in the number of normal (non-cancerous) cells undergoing apoptosis so as to reduce injury to a tissue, an increase in the number of cells undergoing apoptosis when hyperproliferation is at least partially responsible for a condition or disease, and a decrease in the severity or frequency of side effects from a therapeutic agent, decrease in hyperproliferation, reduction in tumor growth, delay of metastases, and reduction in cancer cell or tumor cell proliferation rate. The amount of active compound administered will depend, for example, on the subject being treated, the subject's weight, the manner of administration, the severity of the condition, the specific chemotherapeutic agent utilized, and the judgment of the prescribing physician.

Any embodiment of the invention can include pharmaceutically acceptable salts of compounds 1-18 of Scheme 1. The salts can be prepared from pharmaceutically acceptable non-toxic acids. Also, any embodiment of the invention can include one or a combination of compounds 1-18 of Scheme 1.

The present invention may be better understood by referring to the accompanying examples, which are intended for illustration purposes only and should not in any sense be construed as limiting the scope of the invention.

### Example 1

Glioblastoma multiforme is an extremely aggressive and invasive form of central nervous system (CNS) tumor commonly treated with the chemotherapeutic drug Temozolomide (TMZ). Unfortunately, the median survival is still less than 18 months. In this study, we test the anti-tumor capability of a phenanthroline-based ligand, 2,9-di-sec-butyl-1,10-phenanthroline (SBP).

In an effort to assess the anti-tumor capabilities of SBP, in vitro and in vivo studies were considered using proliferative GL-26 glioma cells. In-vivo studies were done using mice in which a glioma was established by an intracranial injection of GL-26 cells using a stereotactic mouse frame. SBP injections were given intravenously through the retro-orbital route on day 1, 7 and 13 post tumor implantation. Tumor size and SBP toxicity was quantified.

SBP demonstrated strong in-vitro activity against GL-26 cells, with little toxicity towards non-tumorigenic astrocytes. In-vivo experiments demonstrate a significant reduction in tumor growth with administration of SBP alone, with mild toxicity observed in healthy tissues. Furthermore, in-vitro and in-vivo TUNEL stain suggests that SBP induces apoptosis in gliomas.

These experiments suggest SBP is effective in slowing CNS glioblastoma progression and should be considered as a potential compound for future anticancer drug development.

### Introduction

Glioblastoma multiforme is an extremely aggressive and invasive form of central nervous system (CNS) tumor associated with a prognosis of less than one year survival (1-4). Current therapies employ surgical removal in combination with radiation therapy and chemotherapy. Though this removes a large part of the tumor, it often does not eliminate all tumor cells, and relapses occur quickly. Furthermore, chemotherapy and radiation therapy crudely target large regions of the body and can leave the patients with substantial deleterious side effects (5). Temozolomide (TMZ) is a chemotherapeutic drug in use since 1999 to treat advanced glioblastomas and melanomas. Its anti-tumor effects stem from its capability in methylating DNA at the N-7 or O-6 position of guanine, damaging the DNA and thereby causing cell death (1, 3, 6-8). Unfortunately, some patients relapse with TMZ-resistant disease. The resistance to current chemotherapies, limited success of treatment, and poor long term prognosis warrants the search for and creation of new drugs, which alone or in combination with other forms of therapy could target and eradicate tumor cells more efficiently (1, 9).

Gold compounds have been long thought to possess strong anticancer activity, stemming from the fact that initial studies found some gold compounds were able to inhibit HeLa cell growth (10). Unfortunately, gold-based drugs were found to be unstable in-vivo and had no therapeutic advantage over the widely used chemotherapeutic, cisplatin (10, 11). However, the subsequent development of coordinating ligands designed to stabilize gold complexes resulted in the discovery of the anticancer activities of gold(III) polypyridyl complexes, prompting a renewed interest in this area of drug design (11-14). While the development of gold(III) drugs possessing polypyridyl ligand architectures has been progressing, some reports have indicated the polypyridyl ligands themselves exhibit antitumor activity similar to that of the parent gold complex, suggesting that the free ligand may play a role in the activity of this class of gold therapeutics (11, 13). In a recent study of a gold(III) complex bearing the 2,9-di-sec-butyl-1,10-phenanthroline (SBP) ligand, control experiments found that the free SBP ligand exhibited remarkable in-vitro activity against a variety of head-neck and lung tumors (A549 and H1703 lung cancer lines, and 886LN, Tu212, and Tu686 head/neck cancer lines). The study revealed that SBP had in-vitro IC₅₀ values in the nanomolar concentration regime, which were 20-100 times lower than the commonly used chemotherapy cisplatin and 4-14 times lower than the parent gold(III) complex (15). Given that SBP was found to have broad activity against a series tumor cell lines derived from very different human cancers, it was of interest to determine if the drug's general antitumor properties might apply to aggressive glioblastoma tumors.

In the current study, we used a syngeneic mouse model that closely recapitulates the human disease to investigate the anti-tumor capabilities of SBP. This model, using highly proliferative GL-26 glioma cells, allows analysis in an immunologically intact animal where the tumor is tolerated in the murine brain and expresses CD133, a molecule associated with human brain tumors (16, 17). In addition, it is morphologically similar in cell structure, proliferative capacity and infiltrative growth to a human malignant glioma. As such, the model closely mimics the proliferation of glioblastoma in humans (2, 17-19). Our data demonstrate improved and significant in-vitro activity against GL-26 cells compared to TMZ, and initial studies indicate that SBP induces cell death and not cell cycle arrest. Furthermore, in-vivo experiments demonstrate a significant reduction in tumor growth with administration of SBP as an individual drug, with minimal pathology observed in healthy tissues. These experiments suggest SBP is effective in slowing CNS glioblastoma progression and should be considered as a potential compound for future anticancer drug development.

### Materials and Methods

Compound Synthesis:

2,9-di-sec-butyl-1,10-phenanthroline (SBP) and 2-sec-butyl-1,10-phenanthroline (monosecbutyl-phenanthroline; MSBP) were synthesized and purified according to previously reported protocols (20, 21).

Cell Lines:

The murine (C57BL/6) glioma cell line, GL-26, which is highly tumorigenic in the C57BL/6 mice. GL-26 cells were cultured in DMEM/F12 supplemented with 10% FCS, 1% penicillin/streptomycin, 1% L-glutamine and 1% non-essential amino acids. Human foreskin fibroblasts (HFFs) were cultured in DMEM/F12 supplemented with 10% FCS and 1% penicillin/streptomycin. Primary murine astrocytes were purified from C57BL/6 neonate brains and cultured in DMEM/F12 supplemented with 10% FCS, 1% non-essential amino acids, 1% Lglutamine, 50 IU/ml penicillin, 50 mg/ml streptomycin and 10 mM Hepes buffer.

Growth Assay:

The sulforhodamine B (SRB) cytotoxicity assays were adapted from Skehan et al. (22). Briefly, either HFF, primary astrocytes or GL-26 cells were plated in 96-well plates at a density of 4,000 cells/well in a volume of 1004 overnight at 37° C. and 5% CO₂. DMSO stock solutions of SBP, MSBP or TMZ were used at a concentration range of 0.1-25 μM for 48 hr before the supernatant was discarded and the cells were fixed for 1 hr with 10% cold trichloroacetic acid (100 μL, per well). Cells used in recovery assay received fresh media for 48 hrs following the 48 hr drug incubation. The plate was then washed 5 times with de-ionized water, air dried, and stained with 0.4% SRB for 10 min (50 μL, per well). After washing 5 times in 1% acetic acid and air-drying, bound SRB was dissolved in 10 mM unbuffered Tris base (pH 10.5; 100 μL, per well). Bound SRB was then read by absorbance at 492 nm on a SpectraMax plate reader (Molecular Devices). The percent survival was then calculated based upon the absorbance values relative to control wells (0 μM SBP in 0.1% DMSO).

Propidium Iodide:

GL-26 cells were plated at 4000 cells/well in a 96 well plate in GL-26 media (DMEM/F12 supplemented with 10% FCS, 1% penicillin/streptomycin, 1% L-glutamine and 1% non-essential amino acids). The cells were treated 1 day post-plating with 0.1-25 μM SBP for 48 hours. The cells were then detached with Trypsin/EDTA (Cellgro), washed and resuspended at 500,000 cells/ml in ice cold Na₊/K₊ balanced PBS and fixed by gently adding 70% ethanol and incubating for 2 hrs at 4° C. GL-26 cells were then resuspended in 300-500 μl PI/Triton X-100 staining solution: 10 ml of 0.1% (v/v) Triton X-100 (Sigma) in Na₊/K₊ balanced PBS with 2 mg DNAse-free RNAse A (Sigma) and 0.40 ml of 500 μg/ml PI (Roche). The stain was allowed to incubate at 37° C. for 15 minutes before acquisition on a BD FacsCanto II flow cytometer.

In-Vivo Experiments:

Female C57BL/6 mice were obtained from Jackson Laboratories and maintained in a pathogen free environment under IACUC established protocols at the University of California Riverside. Mice were anesthetized with continuous administration of 2.5% isofluorane. Cultured GL-26 cells were harvested by trypsinization and 90,000 GL-26 cells in 3 μL of sterile Na₊/K₊ balanced PBS were injected intracranially. The injection was administered 1 mm anterior and 2 mm lateral to the junction of the coronal and sagittal sutures (bregma), at a depth of 2 mm using a stereotactic mouse frame. Care was taken in alternating injection order and group assignment (treated vs. non-treated) to assure equal GL-26 cell viability between the two treatment groups. SBP was administered intravenously through the retro-orbital route at a concentration of 10 mg/kg in 200 μL sterile Na₊/K₊ balanced PBS. Drug was administered one, seven, and 13 days after tumor implantation.

Histology: For brain tumor histology, mice were perfused intracardially with 4% formaldehyde in Na₊/K₊ balanced PBS and brains were extracted, incubated in 4% formaldehyde overnight followed by 30% sucrose in Na₊/K₊ balanced PBS. Brains were flash frozen in isopentane, embedded in optimal cutting temperature compound (OCT), cryosectioned in coronal sections (12 μm) and stained with hematoxylin and eosin. Another cohort of equivalently drug-treated mice was used for liver, lung and gut histology. The mice were then sacrificed on day 19, and the liver, lung and gut tissues were collected and placed in 4% formaldehyde in Na₊/K₊ balanced PBS overnight. The organs were then placed for 48 hrs in 70% EtOH before paraffin embedding and sectioning at 6 μm. Sections were then stained with hematoxylin and eosin and pathology was assessed blindly and independently by a trained pathologist. A TUNEL staining kit was obtained from TREVIGEN (NeuroTACS II In Situ Apoptosis Detection Kit, Cat#4823-30-K) and used for both ex-vivo slices and in-vitro staining according to manufacturers instructions.

Liver Toxicity:

Intra-cardial blood was collected from the non-tumor bearing mice and subjected to centrifugation for 10 minutes to collect the serum. Aspartate transaminase and Alanine transaminase levels were measured in the serum using Bio Scientific (3913 Todd Lane Suite 312 Austin, Tex.) colorimetric kits (Cat#5605-01 and 3460-08 respectively).

### Results

### The Compound SBP Inhibits Glioma Cell Growth In-Vitro

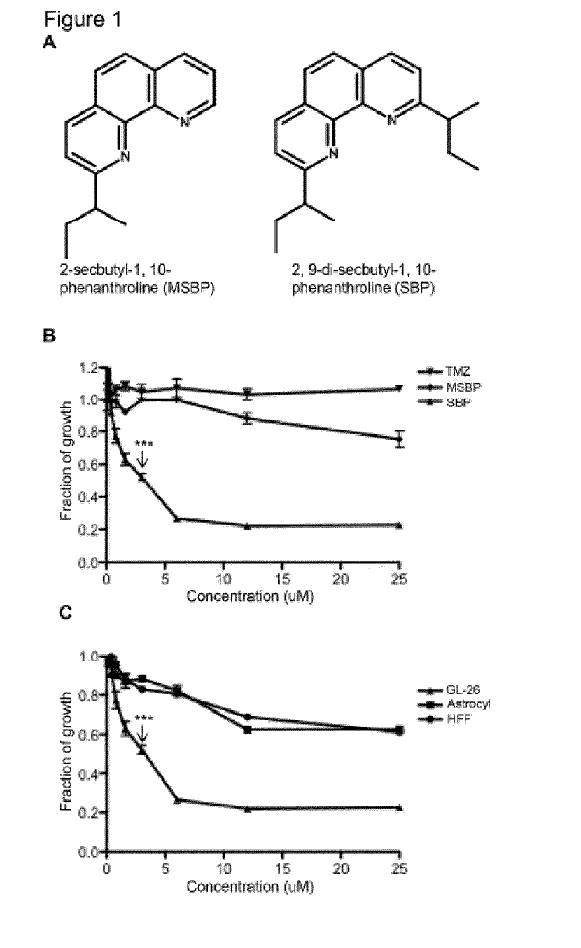

To determine if SBP has the capacity to inhibit the growth of the aggressive GL-26 glioma cell line, cells were cultured in-vitro and incubated with concentrations of SBP and TMZ from 0.1 uM to 25 uM for 48 hours. The drug was then removed and the effect of SBP on cell number was assessed using the Sulforhodamine B colorimetric assay (SRB) assay. A dose related decrease in cell number between 0.8 and 6 μM was observed in cells treated with SBP, whereas TMZ did not change cell number at any tested concentrations. At the IC₅₀ of SBP (3 μM), MSBP and TMZ activity are significantly lower (p<0.0001 and p=0.0005, respectively). Higher concentrations of SBP (6 uM to 25 uM) inhibited GL-26 cell growth 66±1% and 67±0.5% respectively, whereas MSBP inhibited 0.6±1.2% and 24.7±5% respectively. TMZ failed to inhibit cell growth at these concentrations (FIG. 1B). Thus at a concentration of 6 μM, SBP represents a 66% improvement over the current recommended drug TMZ.

In order to determine the toxicity window of the drug between tumor cells and normal cells, murine primary astrocytes and human foreskin fibroblasts (HFF) were treated with SBP (0.1-25 uM) for 48 hours and toxicity was assessed using SRB. At concentrations of up to 25 uM, astrocyte and HFF growth was inhibited by 37.3±0.9% and 36.9±1.3% compared to 67%±0.5% inhibition of GL-26 cells (FIG. 1C). The optimal concentration registered at 6 μM with GL-26 cells inhibited astrocyte growth by 18%, HFF growth by approximately 20%, and GL-26 growth by 66%. At the IC₅₀ of SBP (3 μM) treated GL-26 cells, the growth of equally treated astrocytes and HFF is significantly greater (p=0.0001 and p=0.0002, respectively). These results suggest SBP is significantly more effective at inhibiting GL-26 tumor cell growth than the currently used glioma chemotherapy TMZ, with significantly lower toxicity to non-tumorous cells.

### The Compound SBP Induces GL-26 Apoptosis

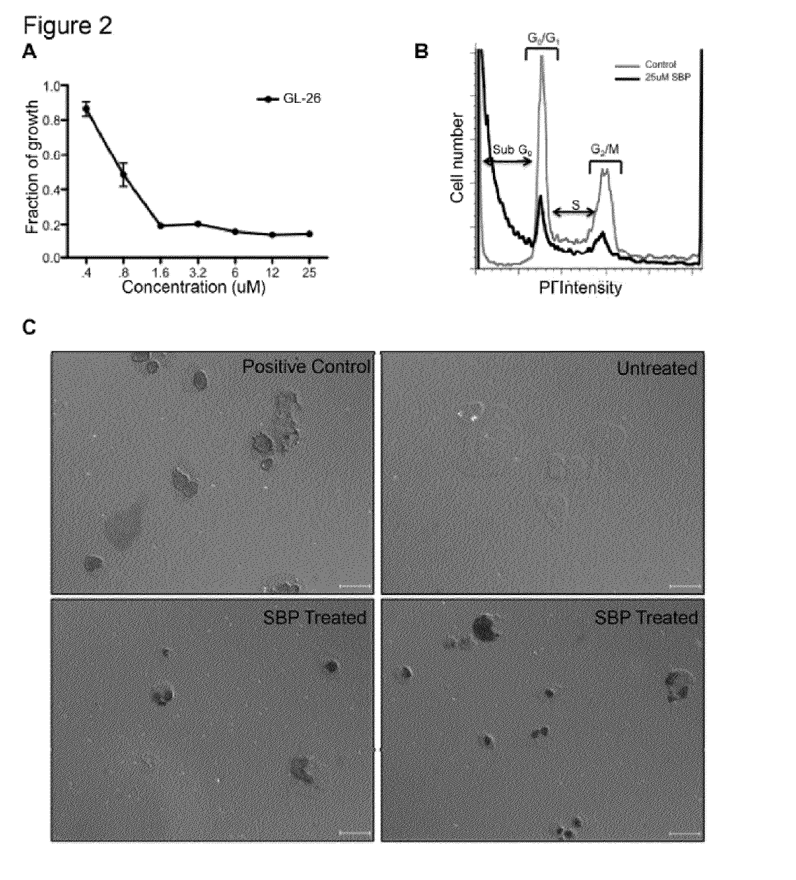

To test if constant SBP administration is necessary to maintain growth inhibition, an SRB recovery assay was performed where GL-26 cells were plated and treated as above, but SBP was removed after a 48-hour incubation and replaced by fresh media. The cells were allowed to grow for an additional 48 hours before performing the SRB viability assay. Allowing the glioma cells to recover for an additional 48 hrs in fresh media did not rescue them, but rather significantly greater cell death was observed in the treated wells. Indeed, although at 0.8 mM SBP mildly inhibit GL-26 cell growth, this sharply increases to 31.5±6.6% 48 hours after the completion of treatment. At higher concentrations of SBP (1.6-25 mM), cell growth was further inhibited (up to 85.4±0.2%) by the extra incubation period (FIG. 2A). These data demonstrate that GL-26 cells continue dying after the removal of SBP and suggests that SBP targets and disrupts cell survival rather than cell proliferation mechanisms.

To directly assess effects on proliferation versus apoptosis, cell cycle analysis was performed using propidium iodide. FIG. 2B demonstrates that SBP treated cells are still capable of advancing to the S, G₂ and mitotic phases, and are thus not arrested in the G₁ phase. However, the proportion of cells in these phases is less and treated cells have a significantly larger population of dead cells compared to untreated controls.

To test if cell death is a result of apoptosis, cultured GL-26 cells treated with SBP were stained with an apoptosis detection kit (FIG. 2C). The dark nucleic staining indicates apoptosis in the nuclease-treated positive control and the SBP treated GL-26 cells. No dark nucleic stain was detected in the entire untreated sample, suggesting that no apoptosis occurred during the culturing of GL-26 cells in media alone (FIG. 2C). These results suggest that SBP does not affect cell cycle progression, but rather kills GL-26 cells by inducing apoptosis.

### SBP Inhibits In-Vivo Glioma Growth

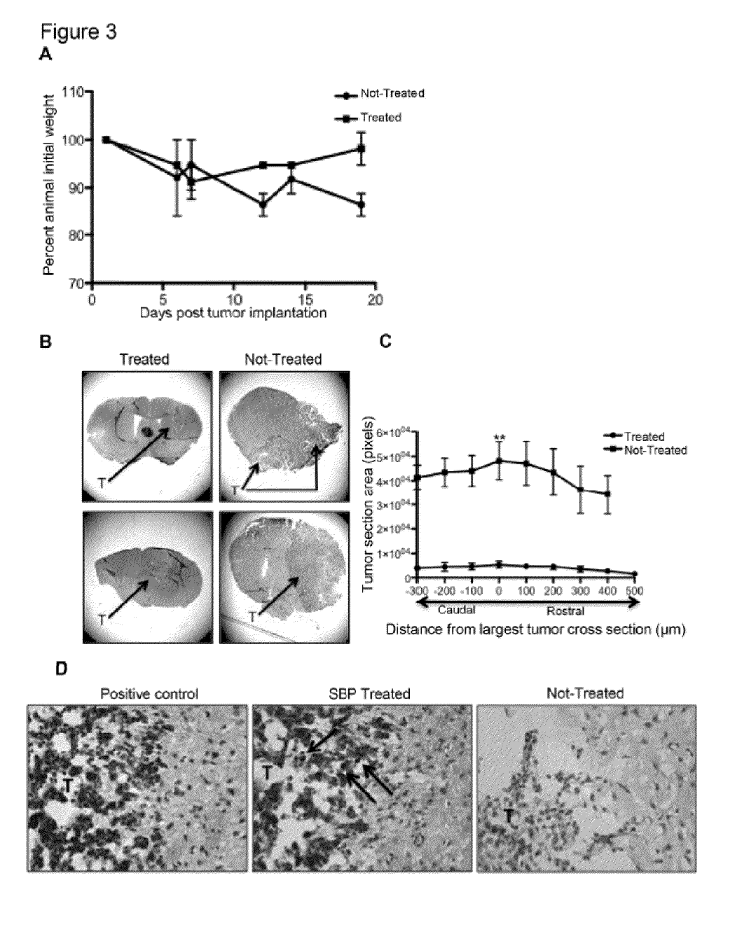

The GL-26 cell line most accurately models human glioblastoma multiforme (GBM), as this cell line is extremely aggressive and mice routinely die within 30 days following intracranial injection. To test if SBP is capable of inhibiting the growth of a tumor in-vivo, mice were injected intracranially with 90,000 GL-26 cells at day 0. This type of tumor implantation has been shown to lead to an aggressive tumor within a week (2, 17, 23). Mice were then treated intravenously with SBP or saline solution on days 1, 7 and 13 post tumor cell injection. Body weights were recorded for the 19 days after tumor implantation, and as expected, a decrease in weight was observed in both treated and non-treated groups during the first week post tumor implantation (FIG. 3A) (2). However, whereas mice treated with SBP regained 98.1±3.4% of their initial weight, mice left untreated exhibited continued weight loss (FIG. 3A). A best-fit line (not shown) revealed that the non-treated group significantly deviated from zero (p=0.0261) whereas the SBP treated mice weights did not (p=0.8792).

Treated and untreated mice were sacrificed at day 19 and serial brain sections were stained with hematoxylin and eosin to reveal general morphology. In untreated mice, tumors were extensive. Glioma growth expanded from the striatum to most of the cortex of the injected hemisphere (FIG. 3B). In contrast, mice treated with SBP revealed noticeably smaller tumors than the untreated group (FIG. 3C). Indeed, tumors in the SBP group seemed to be restricted to areas directly adjacent to the needle tract, constrained to small areas of the striatum, sometimes expanding minimally to the cortex. In some cases, the untreated tumors expanded to the contralateral hemisphere, whereas the treated tumors were not observed to penetrate this region (FIG. 3B). For each animal, tumor size was quantified by pixel area starting at the largest tumor cross-section (position 0) and measuring the tumor area in 100 μm intervals (rostral and caudal). Untreated animals were found to possess significantly larger tumors than the SBP treated group (Not treated: 48187±7736, SBP treated: 5489±1369, p=0.0056, measured at the largest tumor cross-section) (FIG. 3C).

The in-vitro assays suggested that SBP inhibits GL-26 cell growth via the induction of apoptosis. To test if this was occurring in-vivo, in situ TUNEL staining was conducted on serial brain sections from treated and untreated mice (FIG. 3D). The untreated tumor displays feint positive TUNEL stain not inconsistent with tumor growth and destruction of tissue (8). However, in contrast to the control sections, SBP treated tumors revealed dark brown cytoplasmic and nuclear staining indicative of cell necrosis and increased DNA fragmentation, respectively (FIG. 3D). This suggests that SBP strongly inhibits tumor growth resulting in reduced CNS damage. This process is thought to occur in part by tumor cell apoptosis, although further experimentation is required to more clearly identify the mechanism of action. Overall, SBP treated mice appear healthier and gain their original weight back within a couple of weeks following tumor injection.

SBP does not Cause Overt Peripheral Pathology

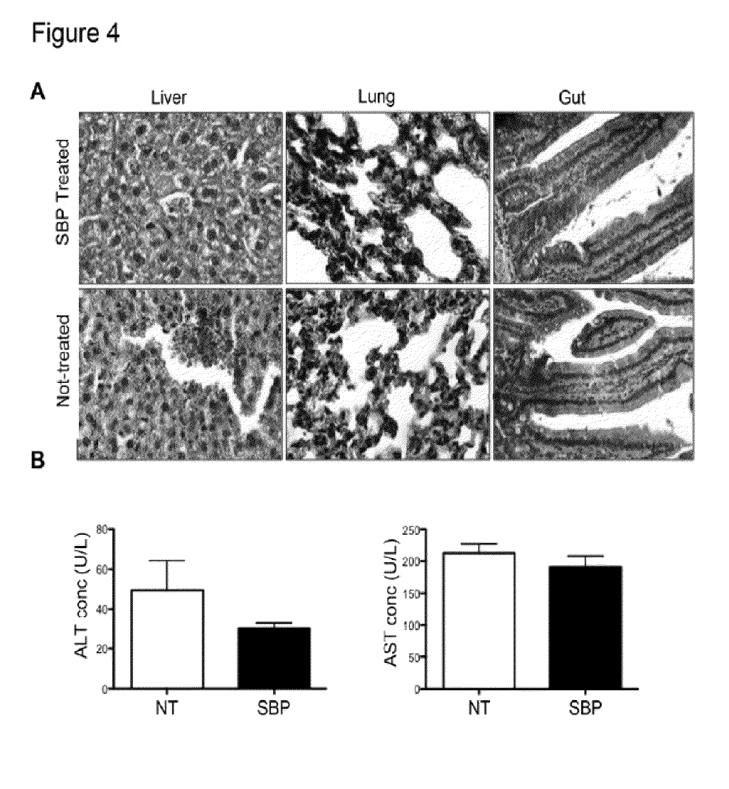

To test if the low level of growth inhibition noted in HFF cells translated into lower systemic toxicity in-vivo, non-tumor bearing control and treatment groups of mice were treated with saline and SBP (10 mg/kg), respectively, on days one, seven, and 13. On day 19 following the first SPB or saline injection, the liver, lungs and proximal small intestine were collected for histopathological analysis. In the duodenum, no general pathology that might have resulted from inhibition of cell division was detected, as there was no change in crypt or villus architecture, or goblet cell density between treated and untreated mice. Analysis of the lung tissue also revealed no overt pathology. Treated liver sections revealed some endothelial damage, but no overt hepatocyte damage (FIG. 4A).

Serum concentrations of liver enzymes were also measured to provide an indication of toxicity (24, 25). Neither aspartate transaminase (AST) nor alanine transaminase (ALT) concentrations, an early indication of toxicity by an intravenously administrated drug (24-26), were significantly different between treated and non-treated mice (ALT: 49.71±14.61 U/L and 30.21±2.73 U/L p=0.2596; AST: 212.3±14.62 U/L and 191.3±16.74 U/L respectively p=0.3982) (FIG. 4B). This suggests minimal toxicity is observed in-vivo after treatment with SBP at the same concentrations and duration as are effective against the glioblastoma.

### DISCUSSION

With current combination therapies extending the survival rate of patients with gliomas by a median of 8-18 months (3, 9), more potent compounds are desperately needed to control tumor growth. In this study, we used the GL-26 cell line as a model for glioblastoma. The GL-26 cells express the mouse version of the human glioma associated CD133 molecule (16, 17), and exhibit similar aggressive, proliferative and tumorigenic properties as gliomas seen in humans. Indeed, when injected into the mouse striatum, the GL-26 cells establish a large and rapidly growing tumor (4, 5, 16-18, 23). This is therefore a good model for testing possible anti-tumor compounds both in-vitro and in-vivo.

Given the remarkable in-vitro efficacy recently reported for SBP against a variety of head/neck and lung tumor cell lines (15), we sought to test the in-vitro anticancer activity of SBP against the GL-26 cell line and compare it to the clinically used glioblastoma drug TMZ. Even though the in-vitro activity of polypyridyl ligands such as SBP has been previously reported (11-14), this report is to our knowledge the first detailed study on the anticancer activity of this class of compounds. We demonstrate strong in-vitro toxicity of SBP against the GL-26 cell line at low micromolar concentrations with a noticeable reduction of tumor size in-vivo.

A major concern in new drug development is the side effects associated with the drug and more importantly, the toxicity towards non-tumor tissue. We have demonstrated high levels of GL-26 death at low micromolar concentrations, with minimal toxicity towards primary astrocytes and the non-tumor HFF cell line. AST and ALT are often used as a marker for liver health during chemotherapy where elevated levels indicate liver damage and the AST/ALT ratio can further be used to differentiate between the causes of liver damage (24-26). Following treatment with SBP, AST and ALT levels were similar to untreated mice and fell well within their respective physiological ranges. Furthermore, histopathalogical analysis of the proximal gut, lung and liver sections only revealed minor damage to liver endothelial cells. These results mirror the low toxicity observed in TMZ-treated patients. Indeed, leukopenia and fatigue are the greatest adverse effects of TMZ treatment, with minimal liver toxicity reported (27-30). In-vivo experiments corroborated in-vitro data demonstrating increased apoptosis in SBP treated groups compared to non-treated. Importantly, SBP treatment resulted in smaller, more contained tumors. This is especially relevant, as contained tumors are easier to remove surgically (31).

The obvious question remains: Is SBP induced apoptosis a result of a similar mechanism as TMZ, which is thought to methylate guanine residues in the DNA, or is it acting via a different mechanism (1, 3, 6-9)? At first glance, it might be expected that SBP acts as a DNA intercalator, as the compound has significant aromatic character. This class of compounds is known to have significant interactions with DNA, which can result in disruption of DNA replication and induction of cell death in a similar fashion to the commonly used chemotherapy cisplatin (32). However, our previous studies indicate that SBP has enhanced antiproliferative effects on cisplatin-resistant cell lines, suggesting that this drug likely initiates tumor cell death via a mechanism not related to DNA interactions (15). To gain further insight about whether SBP might induce tumor cell death via DNA intercalation or by some other mechanism, we determined the in-vitro activity of the structurally analogous 2-sec-butyl-1,10-phenanthroline (MSBP; FIG. 1A) against the GL-26 cell line. It is clear that MSBP has significantly reduced activity compared to SBP (FIG. 1B). This result provides evidence that simply having aromatic character does not result in high antitumor activity, and it is apparent that subtle changes to the alkyl substituents on the phenanthroline backbone significantly change the efficacy of the drug. This further corroborates the conclusion that interactions with DNA are likely not the lone mechanism in which SBP initiates tumor cell death.

One possible alternative explanation for the mechanism of cell death for SBP could be related to the inhibition of intracellular proteins, with one candidate being the protein poly ADP ribose polymerase (PARP). It has been shown that PARP proteins are over expressed after tumor cells are treated with cisplatin, as these proteins are involved in DNA repair mechanisms (33). It has also been recently reported that compounds that can inhibit PARP have antiproliferative effects (33). Given that PARP has a zinc finger domain, SBP could very well chelate the Zn₂₊ cation, which would lead to inhibition of the enzyme's activity. If SBP induces tumor cell death by sequestering an enzyme metal cation in this fashion, this would explain the difference in antitumor activity between SBP and MSBP, as it has been previously shown that changing the alkyl group substitution on the phenanthroline backbone can have significant impact on the metal binding ability of this class of compounds (34). Future studies will therefore aim to determine if SBP indeed acts as a PARP inhibitor, and if this compound induces tumor cell death solely through this mechanism, or by targeting multiple intracellular targets.

In this report, we tested the anti-tumor activity of SBP on glioblastomas both in-vitro and in-vivo. Our data demonstrate the potent anti-tumor activity of SBP in-vitro with minimal toxicity to normal cells. The anti-tumor activity of SBP does not appear to be mediated by cell cycle disruption, but rather by inducing cell death as demonstrated by propidium iodide and TUNEL staining. In-vivo, SBP reduced tumor size without causing apparent pathology to normal tissues, at least within the time-frame of administration used. Though further research should focus on the capability of SBP to stop or eradicate well-established tumors and pinpoint the mechanism of action of this drug, the results described herein clearly demonstrate that SBP has significant anti-glioma activity, thereby making this compound a promising chemotherapy for this aggressive, invasive and difficult to treat class of tumor.

The following publications relating to Example 1 are incorporated by reference herein:
1. Hegi, M. E., A. C. Diserens, S. Godard, P. Y. Dietrich, L. Regli, S. Ostermann, P. Otten, G. Van Melle, N. de Tribolet, and R. Stupp. 2004. Clinical trial substantiates the predictive value of O-6-methylguanine-DNA methyltransferase promoter methylation in glioblastoma patients treated with temozolomide. Clin Cancer Res 10:1871-1874.
2. Safdie, F., S. Brandhorst, M. Wei, W. Wang, C. Lee, S. Hwang, P. S. Conti, T. C. Chen, and V. D. Longo. Fasting enhances the response of glioma to chemo- and radiotherapy. PLoSOne 7:e44603.
3. Poteet, E., G. R. Choudhury, A. Winters, W. Li, M. G. Ryou, R. Liu, L. Tang, A. Ghorpade, Y. Wen, F. Yuan, S. T. Keir, H. Yan, D. D. Bigner, J. W. Simpkins, and S. H. Yang. Reversing the Warburg Effect as a Treatment for Glioblastoma. J Biol Chem.
4. Stupp, R., P. Y. Dietrich, S. Ostermann Kraljevic, A. Pica, I. Maillard, P. Maeder, R. Meuli, R. Janzer, G. Pizzolato, R. Miralbell, F. Porchet, L. Regli, N. de Tribolet, R. O. Mirimanoff, and S. Leyvraz. 2002. Promising survival for patients with newly diagnosed glioblastoma multiforme treated with concomitant radiation plus temozolomide followed by adjuvant temozolomide. J Clin Oncol 20:1375-1382.
5. Sughrue, M. E., I. Yang, A. J. Kane, M. J. Rutkowski, S. Fang, C. D. James, and A. T. Parsa. 2009. Immunological considerations of modern animal models of malignant primary brain tumors. Journal of translational medicine 7:84.
6. Mutter, N., and R. Stupp. 2006. Temozolomide: a milestone in neuro-oncology and beyond? Expert Rev Anticancer Ther 6:1187-1204.
7. Srivastava, D. K., B. J. Berg, R. Prasad, J. T. Molina, W. A. Beard, A. E. Tomkinson, and S. H. Wilson. 1998. Mammalian abasic site base excision repair. Identification of the reaction sequence and rate-determining steps. J Biol Chem 273:21203-21209.
8. Kim, T. G., C. H. Kim, J. S. Park, S. D. Park, C. K. Kim, D. S. Chung, and Y. K. Hong. Immunological factors relating to the antitumor effect of temozolomide chemoimmunotherapy in a murine glioma model. Clin Vaccine Immunol 17:143-153.
9. Chamberlain, M. C., M. J. Glantz, L. Chalmers, A. Van Horn, and A. E. Sloan. 2007. Early necrosis following concurrent Temodar and radiotherapy in patients with glioblastoma. J Neurooncol 82:81-83.
10. Thang, D. C., P. Jacquignon, and M. Dufour. 1976. Synthèse d′amines hétérocycliques, cancérostatiques potentiels. Journal of Heterocyclic Chemistry 13:641-644.
11. Wein, A. N., A. T. Stockhausen, K. I. Hardcastle, M. R. Saadein, S. B. Peng, D. Wang, D. M. Shin, Z. G. Chen, and J. F. Eichler. Tumor cytotoxicity of 5,6-dimethyl-1,10-phenanthroline and its corresponding gold(III) complex. J Inorg Biochem 105:663-668.
12. Palanichamy, K., N. Sreejayan, and A. C. Ontko. Overcoming cisplatin resistance using gold(III) mimics: anticancer activity of novel gold(III) polypyridyl complexes. J Inorg Biochem 106:32-42.
13. Messori, L., F. Abbate, G. Marcon, P. Orioli, M. Fontani, E. Mini, T. Mazzei, S. Carotti, T. O'Connell, and P. Zanello. 2000. Gold(III) complexes as potential antitumor agents: solution chemistry and cytotoxic properties of some selected gold(III) compounds. J Med Chem 43:3541-3548.
14. Shi, P., Q. Jiang, Y. Zhao, Y. Zhang, J. Lin, L. Lin, J. Ding, and Z. Guo. 2006. DNA binding properties of novel cytotoxic gold(III) complexes of terpyridine ligands: the impact of steric and electrostatic effects. J Biol Inorg Chem 11:745-752.
15. Sanghvi, C. D., P. M. Olsen, C. Elix, S. Peng, D. Wang, Z. Chen, D. M. Shin, K. I. Hardcastle, J. Eichler F, and C. MacBetha E. 2013. Antitumor Properties of Five-Coordinate Gold(III) Complexes Bearing Substituted Polypyridyl Ligands. Journal of Inorganic Biochemistry.
16. Golebiewska, A., S. Bougnaud, D. Stieber, N. H. Brons, L. Vallar, F. Hertel, B. Klink, E. Schrock, R. Bjerkvig, and S. P. Niclou. Side population in human glioblastoma is nontumorigenic and characterizes brain endothelial cells. Brain.
17. Candolfi, M., J. F. Curtin, W. S. Nichols, A. G. Muhammad, G. D. King, G. E. Pluhar, E. A. McNiel, J. R. Ohlfest, A. B. Freese, P. F. Moore, J. Lerner, P. R. Lowenstein, and M. G. Castro. 2007. Intracranial glioblastoma models in preclinical neuro-oncology:neuropathological characterization and tumor progression. J Neurooncol 85:133-148.
18. Smith, K. E., S. Fritzell, W. Badn, S. Eberstal, S. Janelidze, E. Visse, A. Darabi, and P. Siesjo. 2009. Cure of established GL261 mouse gliomas after combined immunotherapy with GM-CSF and IFNgamma is mediated by both CD8+ and CD4+ T-cells. Int J Cancer 124:630-637.
19. Sughrue, M. E., I. Yang, A. J. Kane, M. J. Rutkowski, S. Fang, C. D. James, and A. T. Parsa. 2009. Immunological considerations of modern animal models of malignant primary brain tumors. J Transl Med 7:84.
20. Pallenberg, A. J., K. S. Koenig, and D. M. Barnhart. 1995. Synthesis and Characterization of Some Copper(I) Phenanthroline Complexes. Inorganic Chemistry 34:2833-2840.
21. Jakobsen, S. r., and M. Tilset. A rapid synthesis of asymmetric alkyl- and aryl-2,9-disubstituted 1,10-phenanthrolines. Tetrahedron Letters 52:3072-3074.
22. Skehan, P., R. Storeng, D. Scudiero, A. Monks, J. McMahon, D. Vistica, J. T. Warren, H. Bokesch, S. Kenney, and M. R. Boyd. 1990. New colorimetric cytotoxicity assay for anticancer-drug screening. J Natl Cancer Inst 82:1107-1112.
23. David, C., J. P. Nance, J. Hubbard, M. Hsu, D. Binder, and E. H. Wilson. Stabilin-1 expression in tumor associated macrophages. Brain Res 1481:71-78.
24. Amacher, D. E. 1998. Serum transaminase elevations as indicators of hepatic injury following the administration of drugs. Regul Toxicol Pharmacol 27:119-130.
25. Danan, G., and C. Benichou. 1993. Causality assessment of adverse reactions to drugs—I. A novel method based on the conclusions of international consensus meetings: application to drug-induced liver injuries. J Clin Epidemiol 46:1323-1330.
26. Scheig, R. 1996. Evaluation of tests used to screen patients with liver disorders. Prim Care 23:551-560.
27. Pouratian, N., J. Gasco, J. H. Sherman, M. E. Shaffrey, and D. Schiff. 2007. Toxicity and efficacy of protracted low dose temozolomide for the treatment of low grade gliomas. J Neurooncol 82:281-288.
28. Niewald, M., C. Berdel, J. Fleckenstein, N. Licht, R. Ketter, and C. Rube. Toxicity after radiochemotherapy for glioblastoma using temozolomide—a retrospective evaluation. Radiat Oncol 6:141.
29. Su, Y. B., S. Sohn, S. E. Krown, P. O. Livingston, J. D. Wolchok, C. Quinn, L. Williams, T. Foster, K. A. Sepkowitz, and P. B. Chapman. 2004. Selective CD4+ lymphopenia in melanoma patients treated with temozolomide: a toxicity with therapeutic implications. J Clin Oncol 22:610-616.
30. Friedman, H. S., T. Kerby, and H. Calvert. 2000. Temozolomide and treatment of malignant glioma. Clin Cancer Res 6:2585-2597.
31. Duffau, H. 2009. A personal consecutive series of surgically treated 51 cases of insular WHO Grade II glioma: advances and limitations. J Neurosurg 110:696-708.
32. Kelland, L. R. 2006. The Development of Orally Active Platinum Drugs. In Cisplatin. Verlag Helvetica Chimica Acta. 497-521.
33. Mendes, F., M. Groessl, A. A. Nazarov, Y. 0. Tsybin, G. Sava, I. Santos, P. J. Dyson, and A. Casini. Metal-based inhibition of poly(ADP-ribose) polymerase—the guardian angel of DNA. J Med Chem 54:2196-2206.
34. Satterfield, M., and J. S. Brodbelt. 2001. Relative binding energies of gas-phase pyridyl ligand/metal complexes by energy-variable collisionally activated dissociation in a quadrupole ion trap. Inorg Chem 40:5393-5400.

### Example 2

### Antitumor Activity of a Polypyridyl Chelating Ligand in Combination with a Platinum-Based Chemotherapy Drug: In-Vitro Inhibition of Glioma

### Protocol

Compound Synthesis:

2,9-di-sec-butyl-1,10-phenanthroline (SBP) was synthesized and purified as previously described.

Cell Line:

The murine (C57BL/6) glioma cell line, GL-26, which is highly tumorigenic in C57BL/6 mice, were used for these in-vitro experiments. GL-26 cells were cultured in DMEM/F12 supplemented with 10% FCS, 1% penicillin/streptomycin, 1% L-glutamine and 1% non-essential amino acids.

Growth Assay:

Sulforhodamine B (SRB) cytotoxicity assays were utilized to assess antitumor activity. GL-26 cells were plated in 96 well plates at a density of 4,000 cells/well (in a total volume of 1004) and incubated for 24 hours in a humidified atmosphere of 5% CO2 at 37° C. GL-26 cells were then exposed to Cisplatin at 0-25 μM with either 0.1 or 0.4 μM SBP for 48 hours. The supernatant was then discarded and the GL-26 cells were fixed with 10% cold trichloroacetic acid for 1 hour (100 μL/well). The cells were washed 5 times with de-ionized water, air dried, and stained with 0.4% SRB for 10 min (50 μL/well). GL-26 cells were then washed 5 times in 1% acetic acid and allowed to air dry. The bound SRB was dissolved in a 10 mM unbuffered Tris base (pH 10.5; 100 μL per well) and quantified by the absorbance at 492 nm using the SpectraMax plate reader (Molecular Devices). The fraction of cell growth was determined based upon the absorbance values in comparison to the control wells (0 μM SBP in 0.5% DMSO). All cell growth assays were done in a minimum of triplicates.

### Results

GL-26 cells were cultured in-vitro and exposed to 0-25 μM Cisplatin with either 0.1 or 0.4 μM SBP for 48 hours to determine if the polypyridyl ligand in combination with the platinum-based chemotherapy drug significantly reduced the growth of GL-26 cells compared to treatment with SBP or Cisplatin alone. Cisplaten is [Pt(NH₃)₂Cl₂] with the following structure:

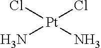

The combination treatment of Cisplatin and SBP appeared to inhibit the growth of GL-26 cells in a dose dependent manner and increased antitumor activity compared to the treatment of GL26 cells with Cisplatin or SBP.

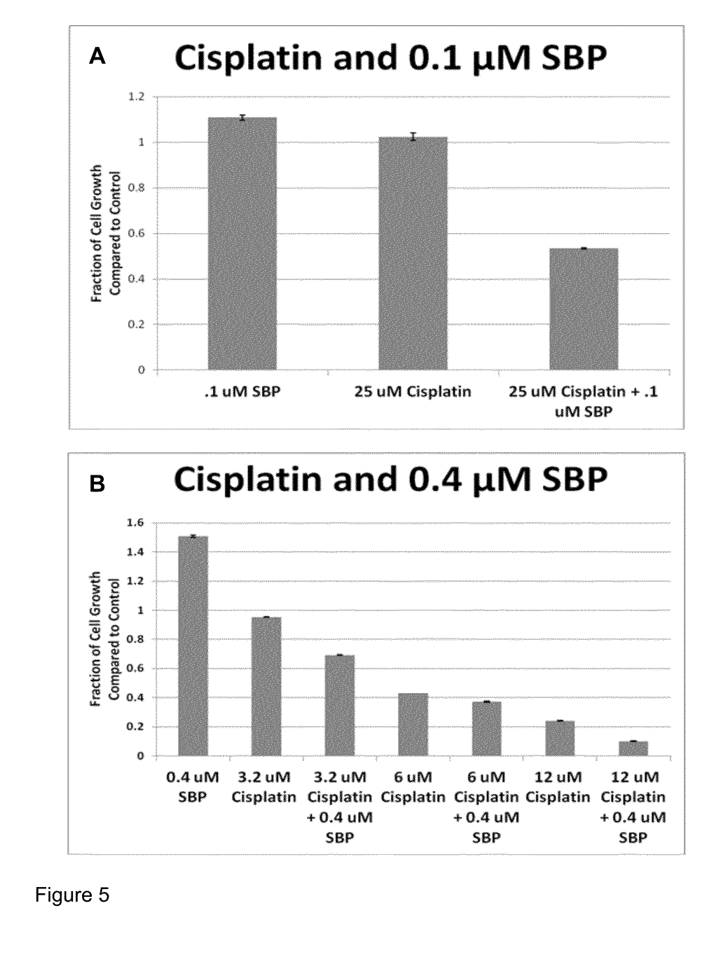

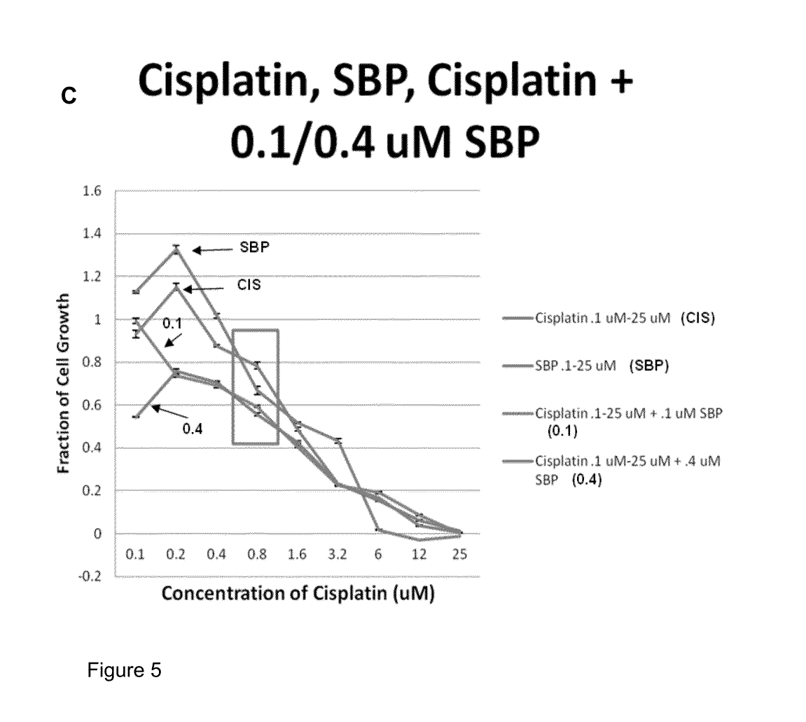

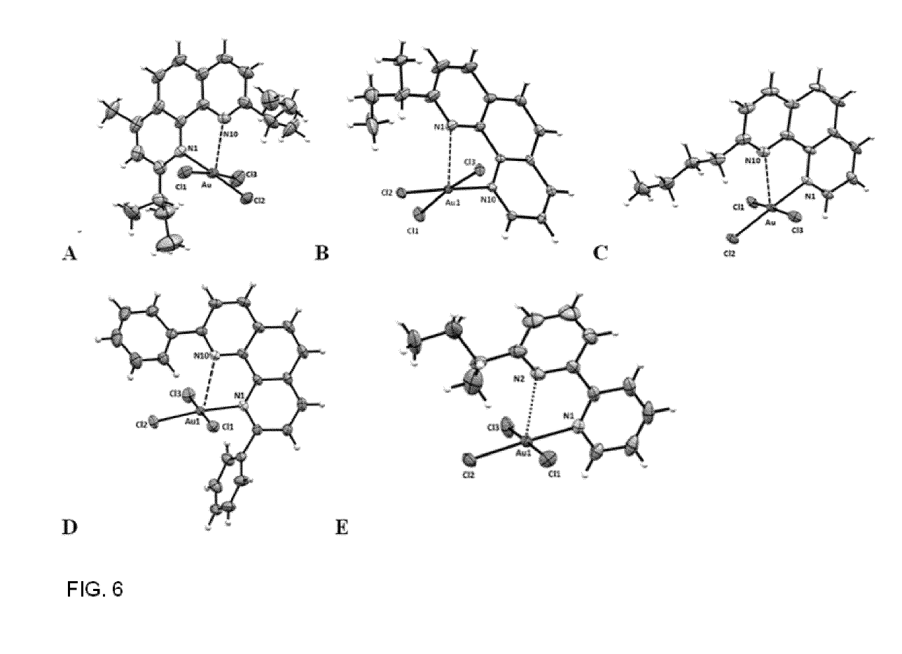

SRB data for the cisplatin/SBP combination therapies is shown in FIG. 5. Fraction of cell growth for treated cells compared to untreated cell cultures is given on the y-axis, and the various concentrations of SBP, cisplatin, and SBP/cisplatin combinations are given on the x-axis. FIGS. 5A and 5B depict fraction of cell growth for specific concentrations of cisplatin/SBP. FIG. 5C depicts the entire growth curve from 0.1-25 μM for the individual SBP, cisplatin, and SBP/cisplatin combination therapies.

The cisplatin/SBP combination therapies show enhanced activity compared to each individual drug treatment, with the 0.8 μM cisplatin showing approximately 78% cell growth compared to untreated tumor cells but 55% cell growth in the presence of 0.1 or 0.4 μM SBP.

### Example 3

### Abstract

Gold(III) complexes bearing bidentate ligands based on the 1,10-phenanthroline and 2,2′-bipyridine scaffolds have shown promising anticancer activity against a variety of tumor cell lines. In particular, our laboratory has previously found that a pseudo five coordinate gold(III) complex possessing the 2,9-di-sec-butyl-1,10-phenanthroline ligand {[(^{di-sec-butyl}phen)AuCl₃]} exhibits antitumor activity against a panel of five different lung and head-neck tumor cell lines. However, the [(^{di-sec-butyl}phen)AuCl₃] complex was determined to be less active than the free 2,9-di-sec-butyl-1,10-phenanthroline ligand. In order to determine if this class of gold(III) complexes has a distinct mechanism of initiating tumor cell death or if these gold complexes simply release the polypyridyl ligand in the intracellular environment, structural analogues of the [(^{di-sec-butyl}phen)AuCl₃] complex have been synthesized and structurally characterized. These structural congeners were prepared by using mono-alkyl and di-phenyl substituted 1,10-phenanthroline ligands, di-alkyl and di-phenyl substituted 4-methyl-1,10-phenanthroline ligands, and mono-alkyl 2,2′-bipyridine ligands. The redox stability of this library of distorted square pyramidal gold(III) complexes has been studied and the in vitro antitumor activity of the gold(III) complexes and corresponding polypyridyl ligands has been determined. The [(^{di-n-butyl}phen)AuCl₃] and [(^{mono-n-butyl}phen)AuCl₃] complexes have been found to be significantly more potent at inhibiting the growth of A549 lung tumor cells than the clinically used drug cisplatin. More importantly, these two gold(III) complexes are significantly more active than their respective free ligands, providing evidence that this class of pseudo five coordinate gold(III) complexes has a mechanism of initiating tumor cell death that is independent of the free ligand.

### 1. INTRODUCTION

Metallotherapeutic drugs have been widely researched for their anti-cancer properties, with cisplatin and its various analogues being the most commonly used to battle tumor cell growth. While cisplatin has been effective in treating a wide variety of cancer in a clinical setting tumors can often develop resistance to cisplatin treatment and patients experience side effects after the administration of the drug [1]. Numerous research groups have pursued alternative metal-based drugs in an attempt to overcome the limitations of cisplatin treatment, and gold(III) complexes represent one class of compounds that have been discovered to possess promising in vitro and in vivo antitumor activity [2-5]. Given the fact gold(III) complexes and complex ions are isoelectronic and often isostructural to platinum(II) drugs, the cytotoxic mechanism for gold(III) complexes was originally thought to be similar to that of cisplatin [6-8]. However, as opposed to targeting DNA and inhibiting DNA replication, gold (III) complexes likely have alternative cellular targets. These include proteins such as thioredoxin reductase (TrxR) [9], zinc finger PARP-1 proteins [PARP=poly(adenosine diphosphate-ribose) polymerase] [10], or cellular proteasomes [11]. These possible alternate mechanisms explain the observation that gold(III) compounds often demonstrate efficacy against cisplatin resistant tumor cell lines, making the design and synthesis of this general class of compounds an attractive area of research [12].

In the course of an ongoing search for possible gold based drugs, a pseudo five coordinate neutral gold (III) complex possessing the 2,9-di-sec-butyl-phenanthroline ligand [(^{di-sec-butyl}phen)AuCl₃] was previously reported in our laboratory [13]. This complex has been shown to have enhanced reduced glutathione (GSH) stability compared to four coordinate square planar complex ions [2] and has also demonstrated significant in vitro anti-tumor activity [13-14]. However it was also observed that the ^{di-sec-butyl}phen ligand has significant anti-tumor activity that is even more pronounced than the corresponding gold complex. Therefore, it was desired to synthesize an expanded library of pseudo five coordinate gold(III) complexes possessing alkyl- and aryl-substituted polypyridyl ligands. This will provide an opportunity to determine if the antitumor activity of this class of gold(III) complexes is dependent on the ligand activity or if these complexes have a distinct mechanism of tumor cell death. We have subsequently synthesized and characterized six new substituted polypyridyl ligands [2,9-di-sec-butyl-1,10-phenanthroline (^{di-sec-butyl}phen), 2-sec-butyl-1,10-phenanthroline (^{mono-sec-butyl}phen), 2,9-di-sec-butyl-4-methyl-1,10-phenanthroline (^{di-sec-butyl-methyl}phen), 2-mono-n-butyl-phenanthroline (^{mono-n-butyl}phen), 2,9-di-phenyl-1,10-phenanthroline (^{di-phenyl}phen), 2,9-di-phenyl-4-methtyl-1,10-phenanthroline (^{di-phenyl-methyl}phen) and 2-mono-sec-butyl-2,2′-bipyridine (^{mono-sec-butyl}bipy) [9] as well as the corresponding gold(III) complexes {[(^{di-sec-butyl}phen)AuCl₃], [(^{mono-sec-butyl}phen)AuCl₃], [(^{di-sec-butyl-methyl}phen)AuCl₃], [(^{mono-n-butyl}phen)AuCl₃], [(^{di-phenyl}phen)AuCl₃], [(^{di-phenyl-methyl}phen)AuCl₃] and [(^{mono-sec-butyl}bipy)AuCl₃]; see Scheme 1}. The previously described [(^{di-methyl}phen)AuCl₃] [15] and [(^{di-n-butyl}phen)AuCl₃] [13] complexes, and their corresponding ligands have also been prepared. Structural characterization of the gold(III) complexes confirms that the unusual pseudo five coordinate geometry previously observed with [(^{di-methyl}phen)AuCl₃] and [(^{di-sec-butyl}phen) AuCl₃] can be broadly accessed via the use of mono- and di-substituted phen and bipy ligands. A description of the general structural parameters of the pseudo five coordinate gold(III) complexes, the stability of these complexes in the presence of the biological reducing agent reduced glutathione (GSH), and a summary of the antitumor efficacy for the entire library of gold(III) complexes and parent alkyl-substituted polypyridyl ligands is summarized herein.

Scheme 1 shows the synthesis of alkyl-substituted 1,10-phenanthroline (^{R}phen) and 6-mono-sec-butyl-2,2′-bipyridine (^{mono-sec-butyl}bipy) ligands, and the corresponding pseudo five coordinate gold(III) complexes.

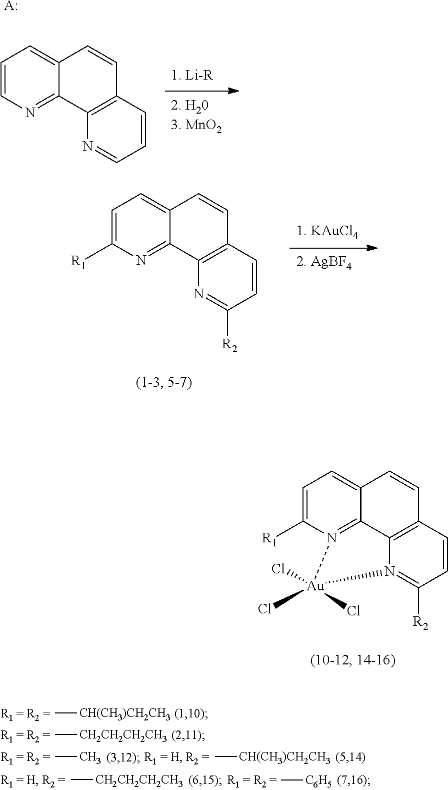

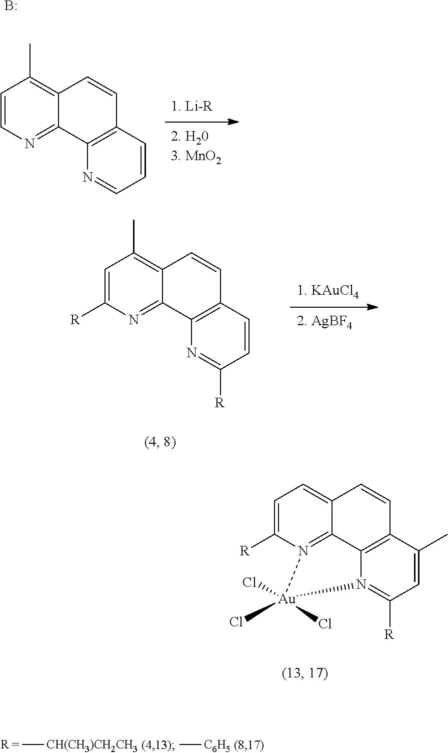

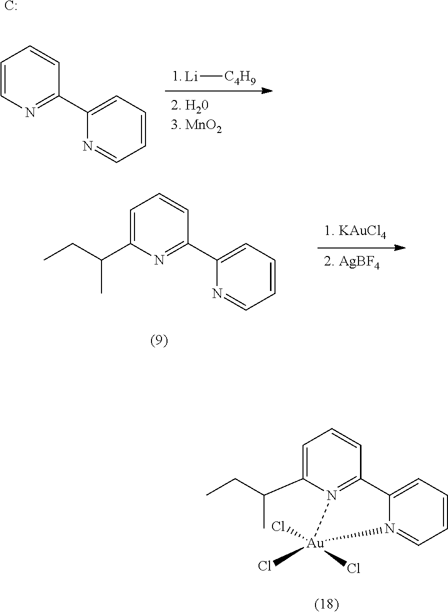

### 2. EXPERIMENTAL PROCEDURES

### 2.1 General Procedures

KAuCl₄.H₂O, AgBF₄, 1,10-phenanthroline, 4-methyl-1,10-phenanthroline, and all solvents were purchased from Sigma Aldrich and used without any further purification. KAuCl₄H₂O and AgBF₄ were stored and weighed out in an inert atmosphere glovebox, and immediately dissolved in acetonitrile solvent before being handled in a chemical fume hood. Aside from limiting the exposure to direct sunlight, no specific handling procedures were taken with compounds 10-18. All cell culturing was done under sterile conditions in a laminar flow hood, and all drugs were dissolved in DMSO and sterifiltered prior to being used in sulforhodamine B (SRB) tumor cells growth assays.

¹H-NMR spectra were obtained using a Varian Inova 300 MHz spectrometer at 20° C., and the chemical shifts were referenced to residual solvent peaks. All UV-Vis spectra were recorded on a Cary 50 UV-Vis spectrophotometer using a 1.0 cm path length quartz cuvette; fluorescence spectra were obtained on a SpectraMax Series fluorescence spectrophotometer; absorbance readings for the SRB assays were collected using a SpectraMax Series microplate reader; mass spectrometry data was obtained using an Agilent 6210 LC-TOF instrument operated in “Multimode” (ESI/APCI; Electrospray Ionization/Atmospheric Pressure Chemical Ionization) with MCP detection; and elemental analyses were carried out by Atlantic Microlabs (Norcross, Ga.).

### 2.2 Synthesis of 2,9-di-sec-butyl-1,10-phenanthroline (^{di-sec-butyl}phen) (1)

Compound 1 was synthesized as previously described [13] by reacting an anhydrous toluene solution of 1,10-phenanthroline (1.00 g, 4.23 mmol) with sec-butyl-lithium (12.84 ml of 1.45 M, 16.92 mmol). The reaction was stirred at 0° C. under an argon atmosphere and the alkyl-lithium reagent was added in a drop-wise fashion over a 20 minute period. This solution was allowed to stir at room temperature for an additional 15 hours under an inert atmosphere. The reaction mixture was then quenched with H₂O (30.0 ml) and the organic layer separated. The aqueous layer was extracted three times with CH₂Cl₂ (20.0 mL), and the combined organic layers were treated with excess MnO₂ (15.0 g). This reaction mixture was stirred overnight and then gravity filtered through celite. This amber filtrate was dried with MgSO₄, gravity filtered, and the solvent was removed by rotary evaporation to yield an amber oil. The crude oil was purified by flash chromatography (basic alumina; 100% hexane wash; elution with 20% CH₂Cl₂ in hexanes) to yield a pale yellow/white solid. ¹H NMR chemical shifts, peak multiplicities and peak integrations, and the UV-vis absorption maxima have been previously reported [13].

### 2.3 Synthesis of 2,9-di-n-butyl-1,10-phenanthroline (^{di-n-butyl}phen) (2)

Compound 2 was synthesized in a similar fashion to compound 1 using 1,10-phenanthroline (0.59 g, 3.26 mmol) and n-butyl-lithium (8.16 ml of 1.6 M in hexanes, 12.94 mmol) [13]. The crude amber oil was purified using flash chromatography (basic alumina; 100% hexane wash; 100% CH₂Cl₂ wash; elution with 4% methanol in CH₂Cl₂) to yield a pale yellow oil. ¹H NMR chemical shifts, peak multiplicities and peak integrations, and UV-vis absorption maxima have been previously reported [13].

### 2.4 Synthesis of 2,9-di-methyl-1,10-phenanthroline (^{di-methyl}phen) (3)

Compound 3 was synthesized in a similar fashion to compound 1 using 1,10-phenanthroline (1.00 g 4.23 mmol) and methyl-lithium(11.9 ml, 2.0 M in hexanes, 16.92 mmol). The crude amber oil was purified using flash chromatography (basic alumina; elution with 20% CH₂Cl₂ in hexanes) to yield a pale yellow oil (35% yield). ¹H NMR (300 MHz, CDCl₃, 20° C.): δ 8.14 (d, 2H), 7.72 (s, 2H), 7.51 (d, 2H), 2.97 (s, 6H). UV-vis λₘₐₓ (ε, M⁻¹ cm⁻¹): 232 nm (46,370), 271 nm (28,597).

### 2.5 Synthesis of 2,9-di-sec-butyl-4-methyl-1,10-phenanthroline (^{di-sec-butyl-methyl}phen) (4)

Compound 4 was synthesized in a similar fashion to compound 1 using 4-methyl-1,10-phenanthroline (1.00 g, 5.15 mmol) and sec-butyl-lithium (14.2 mL of 1.45 M solution, 20.6 mmol). The crude amber oil was purified using flash chromatography (basic alumina; gradient elution using 0-20% CH₂Cl₂:Hexanes) to yield a pale yellow oil (33% yield). ¹H NMR (300 MHz, CDCl₃, 20° C.): δ 8.15 (d, 1H), 7.92 (d, 1H), 7.72 (d, 1H), 7.50 (d, 1H), 7.33 (s, 1H), 3.30 (overlapping m, 4H), 2.76 (s, 3H), 2.01 (m, 2H), 1.83 (m, 2H), 1.46 (overlapping d, 6H), 1.00 (overlapping t, 6H). UV-vis λₘₐₓ (ε, M⁻¹ cm⁻¹): 239 nm (43,720), 275 nm (35,800).

### 2.6 Synthesis of 2-sec-butyl-1,10-phenanthroline (^{mono-sec-butyl}phen) (5)

Compound 5 was synthesized in a similar fashion to compound 1 using 1,10-phenanthroline (1.00 g, 4.23 mmol) and sec-butyl-lithium (3.21 ml of 1.45 M, 4.23 mmol). The crude amber oil was purified using flash chromatography (basic alumina; gradient elution using 0-5% MeOH:CH₂Cl₂), followed by passing the partially purified fraction through a basic alumina plug using pure dichloromethane. A pale yellow oil was obtained (42% yield). ¹H NMR (300 MHz, CDCl₃, 20° C.): δ 9.25 (d, 2H), 8.22 (d, 2H), 8.18 (d, 1H), 7.80 (d, 1H), 7.55 (d, 1H), 3.43 (m, 1H), 2.00 (m, 2H), 1.84 (d, 3H), 0.98 (t, 3H). UV-vis λₘₐₓ (ε, M⁻¹ cm⁻¹): 231 nm (54,760), 268 nm (37,300).

### 2.7 Synthesis of 2-mono-n-butyl-phenanthroline (^{mono-n-butyl}phen) (6)

Compound 6 was synthesized in a similar fashion to compound 1 using 1,10-phenathroline (2.03 g 11.24 mmol) and n-butyl-lithium (6.3 ml, 1.45 M 11.24 mmol). The crude amber oil was purified using flash chromatography (basic alumina; elution with 100% CH₂Cl₂) to yield a pale yellow oil (41% yield). ¹H NMR (300 MHz, CDCl₃, 20° C.): δ 9.24 (d, 1H), 8.24 (d, 1H), 8.16 (d, 1H), 7.75 (m, 2H), 7.61 (dd, 1H), 7.55 (d, 1H), 2.32 (t, 2H), 1.89 (m, 2H), 1.52 (m, 2H), 0.99 (t, 3H). UV-vis λₘₐₓ (ε, M⁻¹ cm⁻¹): 230 nm (16,035), 269 nm (11,544).

### 2.8 Synthesis of 2,9-di-phenyl-1,10-phenanthroline (^{di-phenyl}phen) (7)

Compound 7 was synthesized in a similar fashion to compound 1 using 1,10-phenanthroline (1.00 g, 4.23 mmol) and phenyl-lithium (11.9 ml, 2.0 M). The crude amber oil was purified using flash chromatography (basic alumina; 100% hexane wash; elution with 21% CH₂Cl₂:hexanes) to yield a pale yellow/white solid (38% yield). ¹H NMR (300 MHz, CDCl₃, 20° C.): δ 8.48 (d, 4H), 8.33 (d, 2H), 8.17 (d, 2H), 7.82 (d, 2H), 7.61 (t, 4H), 7.51 (t, 2H). UV-vis λₘₐₓ (ε, M⁻¹ cm⁻¹): 288 nm (17,012), 367 nm (14,705).

### 2.9 Synthesis of 2,9-di-phenyl-4-methtyl-1,10-phenanthroline (^{di-phenyl-methyl}phen) (8)

Compound 8 was synthesized in a similar fashion to compound 1 using 4-methyl-1,10-phenanthroline (1.00 g 5.15 mmol) and phenyl-lithium (10.3 mL, 2.0 M, 20.60 mmol). The crude amber oil was purified using flash chromatography (basic alumina; 100% hexane wash; elution with 20% CH₂Cl₂:hexanes) to yield a pale yellow/white solid (41% yield). ¹H NMR (300 MHz, CDCl₃, 20° C.): δ 8.50-8.45 (m, 3H), 8.31 (d, 1H), 8.17 (d, 1H), 8.01 (d, 2H), 7.84 (d, 1H), 7.63-7.55 (m, 4H), 7.53-7.41 (m, 3H), 2.88 (s, 3H). UV-vis λₘₐₓ (ε, M⁻¹ cm⁻¹): 287 nm (19,406), 316 nm (7,750).

### 2.10 Synthesis of 2-mono-sec-butyl-2,2′-bipyridine (^{mono-sec-butyl}bipy) (9)

Compound 9 was synthesized in a similar fashion to compound 1 using 2,2′ bipyridine (1.50 g 9.60 mmol) and sec-butyl-lithium(6.62 ml, 1.45 M, 9.60 mmol). The crude amber oil was purified using flash chromatography (basic alumina; gradient elution using 5-50% CH₂Cl₂:hexanes) to yield a pale yellow oil (23% yield). ¹H NMR (300 MHz, CDCl₃, 20° C.): δ 8.66 (d, 1H), 8.49 (d, 1H), 8.18 (d, 1H), 7.80 (m, 2H), 7.26 (m, 1H), 7.09 (d, 1H), 2.89 (m, 1H), 1.85 (m, 1H), 1.71 (m, 1H), 1.37 (d, 3H), 0.90 (t, 3H). UV-vis λₘₐₓ (ε, M⁻¹ cm⁻¹): 237 nm (6,013), 284 nm (9,698).

### 2.11 Synthesis of [(^{di-sec-butyl}phen)AuCl₃] (10)

The pseudo five coordinate gold(III) complex was synthesized as previously described [13]. An acetonitrile solution of compound 1 (0.259 g, 0.885 mmol) was added drop wise to an acetonitrile solution of KAuCl₄.H₂O (0.339 g, 0.885 mmol). This reaction mixture was refluxed for 1 hour, during which the solution turned from pale yellow, to red, and finally a bright orange color. An acetonitrile solution of AgBF₄ (0.166 g, 0.885 mmol) was then added drop-wise and the solution immediately became cloudy, and the reaction mixture was stirred overnight under reflux. The solution remained orange in color and a significant amount of colorless/grey precipitate formed. The reaction mixture was gravity filtered through celite and the solvent removed by rotary evaporation. This residual orange solid was dissolved in warm CH₂Cl₂ and extracted three times with deionized water. Finally, the CH₂Cl₂ layer was dried with MgSO₄ and filtered once more through celite. The solution was rotary evaporated, and the orange solid dissolved in a minimum of warm acetonitrile. The complex was recrystallized at −20° C. over three days to form orange crystalline needles. ¹H NMR chemical shifts, peak multiplicities and peak integrations, UV-vis absorption maxima, and elemental analyses have been previously reported [13].

### 2.12 Synthesis of [(^{di-n-butyl}phen)AuCl₃] (11)

Compound 11 was synthesized in an analogous fashion to compound 10 using KAuCl₄.H₂O (0.194 g, 0.490 mmol), compound 2 (0.143 g 0.490 mmol), and AgBF₄ (0.0954 g, 0.490 mmol) [13]. The residual orange solid that was obtained after the reaction workup was recrystallized from acetonitrile at −20° C. over 2 days, yielding orange needles. ¹H NMR chemical shifts, peak multiplicities and peak integrations, UV-vis absorption maxima, and elemental analyses have been previously reported [13].

### 2.13 Synthesis of [(^{di-methyl}phen)AuCl₃] (12)

This compound 12 was previously reported by Robinson and coworkers [15], however it has been synthesized here using a procedure analogous to compound 10 by reacting compound 3 (0.122 g 0.590 mmol), NaAuCl₄.2H₂O (0.230 g 0.59 mmol), and AgBF₄ (0.115 g 0.590 mmol). The residual orange solid that was obtained after the reaction work up was recrystallized from acetonitrile to yield orange needles (52% yield). ¹H NMR (300 MHz, CDCl₃): δ 8.45 (d, 2H), 7.95 (s, 2H), 7.84 (d, 2H), 3.43 (s, 6H). UV-vis λₘₐₓ (ε, M⁻¹ cm⁻¹): 270 nm (33,835), 326 nm (5,959). Elemental analysis (C₁₆H₁₆N₂AuCl₃): Calculated; C=32.87%, H=2.36. Experimental: C=32.41, H=2.24%.

### 2.14 Synthesis of [(^{di-sec-butyl-methyl}phen)AuCl₃] (13)

Compound 13 was synthesized in an analogous fashion to compound 10 using KAuCl₄.H₂O (0.194 g, 0.490 mmol), compound 4 (0.152 g, 0.490 mmol), and AgBF₄ (0.0954 g, 0.490 mmol). The residual orange solid that was obtained after the reaction work up was recrystallized from acetonitrile yielding orange needles (44% yield). ¹H NMR (300 MHz, CDCl₃, 20° C.): δ 8.40 (d, 1H), 8.03 (dd, 2H), 7.82 (d, 1H), 7.64 (s, 1H), 4.66 (m, 1H), 4.38 (m, 1H), 2.93 (s, 3H), 2.01 (m, 2H), 1.97 (m, 2H), 1.57 (overlapping d, 6H), 1.04 (overlapping t, 6H). UV-vis λₘₐₓ (ε, M⁻¹ cm⁻¹): 272 nm (14,781), 320 nm (5,392). Elemental Analysis (C₂₁H₁₆N₂AuCl₃): Calculated; C=41.34%, H=4.30%. Experimental: C=41.47%, H=4.36%.

### 2.15 Synthesis of [(^{mono-sec-butyl}phen)AuCl₃] (14)

Compound 14 was synthesized analogously to compound 10 by reacting KAuCl₄.H₂O (0.339 g, (0.885 mmol), Compound 5 (0.209 g, 0.885 mmol) and AgBF₄ (0.166 g, 0.885 mmol. The residual orange solid that was obtained after the reaction work up was recrystallized from acetonitrile yielding orange needles (54% yield). ¹H NMR (300 MHz, CDCl₃, 20° C.): δ 9.42 (d, 1H), 8.65 (d, 1H), 8.39 (d, 1H), 8.07 (d, 1H), 7.96 (dd, 2H), 7.86 (d, 1H), 3.94 (m, 1H), 2.16 (m, 1H), 1.97 (m, 1H), 1.56 (d, 3H), 1.00 (t, 3H). UV-vis λₘₐₓ (ε, M⁻¹ cm⁻¹): 281 nm (18,060), 322 nm (3,904). Elemental Analysis (C₁₆H₁₆N₂AuCl₃): Calculated: C=35.61, H=2.99. Experimental: C=35.00, H=2.95.

### 2.16 Synthesis of [(^{mono-n-butyl}phen)AuCl₃] (15)

Complex 15 was synthesized analogously to compound 10 by reacting KAuCl₄.H₂O (0.194 g, 0.490 mmol), compound 6 (0.116 g, 0.490 mmol), and AgBF₄ (0.0954 g, 0.490 mmol). The residual orange solid that was obtained after the reaction work up was recrystallized from acetonitrile −20° C. over 3 days, yielding orange needles (78% yield). ¹H NMR (300 MHz, CDCl₃, 20° C.): δ 9.41 (d, 1H), 8.63 (d, 1H), 8.37 (d, 1H), 8.05 (d, 1H), 7.98 (d, 1H), 7.98 (m, 1H), 7.83 (d, 1H), 3.51 (t, 2H), 2.01 (m, 2H), 1.22 (m, 2H), 1.02 (t, 3H). UV-vis λₘₐₓ (ε, M⁻¹ cm⁻¹): 278 nm (23,400), 318 nm (5,840). Elemental Analysis (C₂₄H₁₆N₂AuCl₃): Calculated: C=35.61. H=2.99. Experimental: C=35.84, H=2.92.

### 2.17 Synthesis of [(^{di-phenyl}phen)AuCl₃] (16)

Complex 16 was synthesized analogously to compound 10 by reacting KAuCl₄.H₂O (0.194 g, 0.490 mmol) compound 7 (0.163 g, 0.490 mmol), and AgBF₄ (0.0954 g, 0.490 mmol). The residual orange solid that was obtained after the reaction work up was recrystallized from CH₂Cl₂ and diethyl ether at room temperature yielding red/orange needles (55% yield). ¹H NMR (300 MHz, DMSO, 20° C.): δ 8.61 (d, 2H), 8.17 (d, 4H), 8.11 (d, 4H), 7.59 (overlapping m, 6H). UV-vis λₘₐₓ (ε, M⁻¹ cm⁻¹): 331 nm (9,804), 379 nm (4,792). Elemental Analysis (C₂₄H₁₆N-2AuCl₃): Calculated: C=45.34, H=2.52. Experimental: C=44.49, H=2.54.

### 2.18 Synthesis of [(^{di-phenyl-methyl}phen)AuCl₃] (17)

Complex 17 was synthesized analogously to compound 10 by reacting KAuCl₄.H₂O (0.194 g, 0.490 mmol), compound 10 (0.170 g, 0.490 mmol), and AgBF₄ (0.0954 g, 0.490 mmol). The residual orange solid that was obtained from the reaction work up was recrystallized from CH₂Cl₂ and diethyl ether at room temperature, yielding red/orange needles (yield 22%). ¹H NMR (300 MHz, CDCl₃, 20° C.): δ 9.24 (d, 1H), 8.46 (m, 2H), 8.32 (m, 2H), 8.16 (d, 1H), 8.07 (m, 2H), 7.86 (m, 2H), 7.53 (overlapping m, 5H), 2.87 (s, 3H). UV-vis λₘₐₓ (ε, M⁻¹ cm⁻¹): 306 nm (11,326), 360 nm (3,599), 372 nm (1,517). Elemental Analysis (C₂₅H₁₈N₂AuCl₃): Calculated: C=46.21, H=2.79. Experimental: C=46.28, H=2.67. Mass Spectrometry: Observed M⁺ {[(^{methyl-di-phenyl}phen)AuCl₃]—Cl⁻} [C₂₅H₁₈N₂AuCl₂]⁺=(613.0520 amu); Calculated M⁺ [C₂₅H₁₈N₂AuCl₂]⁺=613.0507 amu.

### 2.19 Synthesis of [(^{mono-sec-butyl}bipy)AuCl₃] (18)

Complex 18 was synthesized analogously to compound 10 by reacting KAuCl₄.H₂O (0.194 g, 0.490 mmol), compound 9 (0.143 g 0.490 mmol), and AgBF₄ (0.0954 g, 0.490 mmol). The residual orange solid that was obtained after the reaction work up was recrystallized from acetonitrile at −20° C. over 2 days, yielding orange needles (45% yield). ¹H NMR (300 MHz, CDCl₃, 20° C.): δ 9.07 (d, 1H), 8.19 (dd, 2H), 7.94 (t, 1H), 7.82 (d, 1H), 7.71 (m, 1H), 7.50 (d, 1H), 3.45 (m, 1H), 1.99 (m, 1H), 1.87 (m, 1H), 1.44 (d, 3H), 0.96 (t, 3H). UV-vis λₘₐₓ, (ε, M⁻¹ cm⁻¹): 284 nm (9,670), 320 nm (3,587), 335 nm (3,360). Elemental Analysis (C₁₆H₁₆N₂AuCl₃): Calculated: C=32.61, H=3.13. Experimental: C=32.50, H=3.08.

### 2.20 X-Ray Studies

Single crystal were coated with paratone oil and mounted on a cryo-loop glass fiber. X-ray intensity data were collected at 100(2) K on a Bruker APEX2 [16] platform-CCD X-ray diffractometer system (fine focus Mo-radiation, λ=0.71073 Å, 50 KV/35 mA power). The CCD detector was placed at a distance of 5.0800 cm from the crystal. A total of 3600 frames were collected for a sphere of reflections (with scan width of 0.3° in ω, starting ω and 2θ angles at −30°, and φ angles of 0°, 90°, 120°, 180°, 240°, and 270° for every 360 frames, 20 sec/frame exposure time). The frames were integrated using the Bruker SAINT software package [17] and using a narrow-frame integration algorithm. Absorption corrections were applied to the raw intensity data using the SADABS program [18]. The Bruker SHELXTL software package [19] was used for phase determination and structure refinement. Direct methods of phase determination followed by two Fourier cycles of refinement led to an electron density map from which most of the non-hydrogen atoms were identified in the asymmetric unit of the unit cell. With subsequent isotropic refinement, all of the non-hydrogen atoms were identified. Atomic coordinates, isotropic and anisotropic displacement parameters of all the non-hydrogen atoms were refined by means of a full matrix least-squares procedure on F². The H-atoms were included in the refinement in calculated positions riding on the atoms to which they were attached. It is noted that [(^{di-phenyl}phen)AuCl₃] (16) was observed to undergo a low temperature phase transition, and [(^{methyl-di-sec-butyl}phen)AuCl₃] (13) and [(^{mono-sec-butyl}phen)AuCl₃] (14) were found to possess disorder in the sec-butyl substituents.

### 2.21 Glutathione Stability Experiments

5.0×10⁻³ M stock solutions of compounds 10-18 were prepared in acetonitrile or DMSO, and subsequently diluted in phosphate buffer (0.10M, pH 7.4) to yield a final gold complex concentration of 5.0×10⁻⁵ M. A stock solution of reduced glutathione in phosphate buffer (0.10M, pH 7.4) was used to add one mole equivalent of GSH to the final gold complex solution, and UV-visible spectra of compounds 10-18 were subsequently collected every hour over a 15 hour period.

### 2.22 Cell Culturing

A549 cells were cultured in RPMI media supplemented with 10% Fetal Bovine Serum and 1% nonessential amino acids. Sub-culturing was carried out every 3-4 days and was completed by removing the supernatant media and treating the adherent cells with 1.0 mL of a 0.25% (w/v) Trypsin/0.5 mM EDTA solution. The cells were then treated for approximately 3 minutes in a 37° C. incubator under a 5% CO₂ atmosphere. Trypsinization was stopped by the addition of 7.0 mL of cell culture media, the suspended cells were collected by centrifugation, and the resulting cell pellet was re-suspended in fresh cell culture media. The cells were then diluted 1:4 by volume and incubated in a 37° C. incubator with 5% CO₂. Cells were grown to 80-90% confluency prior to being used in the SRB colorimetric assays.

### 2.23 SRB Colorimetric Assay

To test the effects of compounds 1-18 on the growth of A549 lung cancer cells, SRB cytotoxicity assays were done as described by Skehan et al. [20] Cells were maintained in RPMI media as described above, collected, and diluted so that cells could be seeded in 96-well plates at a density of 4,000 cells/well. The 96-well plate was incubated overnight at 37° C. under a 5% CO₂ atmosphere. Subsequently, sterifiltered DMSO stock solutions of the drugs were added to the wells in various concentrations (0-25 μM) and the 96-well plate was incubated at 37° C. under a 5% CO₂ for an additional 72 hours. The supernatant cell culture medium was then removed and the cells were fixed for 1 hour with 10% cold trichloroacetic acid (100 μL per well). The trichloroacetic acid was discarded and the plates were washed 5 times with de-ionized water and air dried. After being stained with 0.4% SRB (500 μL per well) and incubated at room temperature for 10 minutes, the cells were washed 5 times with 1% acetic acid and air dried. The bound SRB was dissolved in 10 mM unbuffered Tris, pH 10.5 (1000 μL per well) for 10 minutes at room temperature, and the absorbance at 492 nm was measured using a microplate reader. The percent cell growth was then calculated based upon the absorbance values relative to control samples not containing any drug. Each drug concentration was done in triplicate to yield a percent growth vs. drug concentration curve, and these growth curves were subsequently repeated two additional times. The growth curves from the three experiments were then plotted in GraphPad Prism and best-fit curves were used to generate the IC₅₀ values for each curve. The average IC₅₀ and standard deviation values from these best-fit curves are summarized in Table 3.

### 3. RESULTS AND DISCUSSION

### 3.1 Synthesis and Spectroscopic Characterization

The alkyl-substituted phen ligands have been previously synthesized in our laboratory using a protocol reported by Pallenberg and coworkers [21]. However, a more recent report by Jakobsen and Tilset describes the synthesis of 2-mono- and 2,9-di-alkyl-1,10 phenanthroline (^{R}phen) species using reaction times ranging from 5-40 minutes and with yields ranging from 80-100% [22]. Unfortunately, attempts to reproduce the work by Jackobsen and Tilset were not successful, as incomplete reactions were observed and product mixtures containing significant amounts of starting material were universally obtained. We therefore reverted to using longer reaction times as previously reported by Pallenberg, et al., though it is noted that the use of columns with basic alumina stationary phase was found to improve the ability to isolate pure ^{R}phen ligands compared to the prior use of silica columns. The yields for the purified ^{R}phen ligands reported here range from 20-50%, which are similar to previous reports [13, 21].

The synthesis of a pseudo five coordinate gold(III) complex possessing 2,9-di-methyl,1,10-phenanthroline (^{di-methyl}phen) was previously reported Robinson and coworkers, and was carried out by reacting KAuCl₄ with the ligand in a benzene-methanol solvent mixture [15]. Alternatively, a more recent report by Shaw and Jakobsen describes reacting a mixture of sodium bicarbonate and HAuCl₄.3H₂O in a water/acetonitrile solvent mixture, followed by the addition of alkyl-substituted 2,2′-bipyridine (^{R}bipy) ligands and irradiation in a microwave reactor. This procedure yielded pseudo five coordinate gold(III) complexes possessing ^{di-methyl}bipy, ^{tetra-methyl}bipy, ^{di-methyl}phen, and bi-quinoline ligands [23]. We have previously reported that reacting 2,9-dialkyl-substituted phen ligands in the presence of KAuCl₄, with subsequent addition of AgBF₄ afforded the most convenient route for our laboratory to obtain analogous [(^{R}phen)AuCl₃] complexes [13]. Thus, this synthetic approach was used here to reproduce the previously reported [(^{di-sec-butyl}phen)AuCl₃] (10), [(^{di-n-butyl}phen)AuCl₃] (11), and [(^{di-methyl}phen)AuCl₃] (12) complexes, as well as the new gold(III) complexes possessing mono-substituted phen and bipy ligands, di-phenyl phen ligands, and tri-substituted phen ligands. (compounds 13-18; see Scheme 1). Analytically pure samples of compounds 12-18 were obtained in moderate yields by recrystallization from acetonitrile. The ¹H NMR spectra of complexes 12-18 confirm that the ligands are coordinated to the gold(III) center, evidenced by the downfield shift of the protons on the alkyl substituents and/or the downfield shift of the polypyridyl aromatic protons (see Experimental section). The UV-vis absorption spectra of 12-18 also confirm that the polypyridyl ligands are bound to the gold(III) center, as ligand-to-metal charge transfer (LMCT) bands were observed between 315-380 nm (see Experimental Section). We note that it has been previously observed that the ¹H NMR and UV-vis spectra for non-coordinated [^{R}phenH][AuCl₄] complex ions are not distinguishable from the [(^{R}phen)AuCl₃] complexes [13], therefore elemental analysis of compounds 12-18 was carried out to confirm that the ^{R}polypyridyl ligands directly coordinated to the gold(III) center to form the pseudo five coordinate complexes. These elemental analysis data indeed confirm that the [(^{R}polypyridyl)AuCl₃] complexes were isolated and not [^{R}polypyridylH] [AuCl₄] complex ions (see Experimental section).

### 3.2 X-Ray Crystal Structures of [(^{R}polypyridyl)AuCl₃] Complexes

In order to definitively confirm the pseudo five coordinate geometry of compounds 13-16 and 18, single crystal X-ray diffraction studies were used to structurally characterize this library of gold(III) complexes. Analogous to the previously reported complexes [(di-sec-butylphen)AuCl3] (10), [(di-n-butylphen)AuCl3] (11), and [(di-methylphen)AuCl3] (12), compounds 13-16 and 18 exhibit a distorted square pyramidal geometry (see FIG. 6). This geometry is distinguished by a square planar base comprised of three chloride ligands and one polypyridyl nitrogen donor ligand, with Cl—Au—Cl and Cl—Au—N angles generally ranging from 87-91° (see FIG. 6, and an elongated axial Au—N interaction. This axial gold(III)-nitrogen interaction is longer than a typical Au—N coordinate covalent bond (Au-Naxial distances range from 2.556-2.704 Å; see FIG. 6 and Table 1) yet shorter than the sum of the van der Waal's radii for the two atoms (sum of van der Waal's radii=3.21 Å). This Au-Naxial interaction is also noted for its distinct “lean”, with the Cl—Au-Naxial angles ranging from 108-114° and the Nequatorial-Au-Naxial angles ranging from 71-74° (see FIG. 6 and Table 1). This elongation and distortion of the Au-Naxial bond has been previously discussed in the literature [15] and can be attributed to a combination of the steric interaction between the alkyl/phenyl substituents located adjacent to the nitrogen donor atoms and the proximal chloride ligands, as well as the unfavorable interaction of the axial nitrogen donor lone pair and the electrons located in the dz2 orbital of the gold(III) metal center. The Au—Cl distances (2.26-2.29 Å) and Au-Nequatorial distances (2.05-2.07 Å) for compounds 13-16 and 18 fall within the expected ranges for common gold(III)-chloride and gold(III)-nitrogen coordinate covalent bonds, though it is noted that for compounds 13-16 and 18 the chloride ligands trans to the equatorial nitrogen donor atom have slightly shorter Au—Cl interatomic distances compared to the other two Au—Cl coordinate covalent bonds (see FIG. 6).

In FIG. 6, X-ray crystal structures for compounds 13-16 and 18 are shown. Thermal ellipsoids shown at 50% probability. Notable interatomic distances (Å) and bond angles (°) shown. 6A [(di-sec-butyl-methylphen)AuCl3] (13): Au—N10=2.556(3), Au—N1=2.072(3), Au—Cl1=2.2874(10), Au—Cl2=2.281(10), Au—Cl3=2.2895(11), N1-Au—Cl2=178.14(9), N1-Au—Cl3=89.73, Cl2B—Au—Cl3=90.32(9), N1-Au—Cl1=89.99(9), Cl2-Au—Cl1=90.07(4), Cl3-Au—Cl1=174.66(4), N1-Au—N10=73.51(12), Cl2B—Au—N10=108.34(8). 6B [(mono-sec-butylphen)AuCl3] (14): Au1-N1=2.671(2), Au1-N10=2.0572(17), Au1-Cl1=2.2924(5), Au1-Cl2=2.2778(5), Au1-Cl3=2.2933(5), 10-Au1-Cl2=177.63(5), N10-Au1-Cl3=88.93(5), Cl1-Au1-Cl3=90.095(19), N10-Au1-Cl1=90.06(5), Cl2-Au1-Cl1=90.68(2), Cl3-Au1-Cl1=173.73(2), N1-Au1-N10=71.54(6), Cl2-Au1-N1=110.66(4). 6C [(mono-n-butylphen)AuCl3] (15): Au—N10=2.642(2), Au—N1=2.055(2), Au—Cl1=2.2899(7), Au—Cl2=2.2716(6), Au—Cl3=2.2876(7), N1-Au—Cl2=178.16(6), N1-Au—Cl3=87.18(2), Cl2B—Au—Cl3=90.82(6), N1-Au—Cl1=90.00(3), Cl2-Au—Cl1=90.82(6), Cl3-Au—Cl1=173.76(3), N1-Au—N10=72.24(8), Cl2B—Au—N10=109.49(6). 6D [(mono-sec-butylbipy)AuCl3] (16): Au1-N1=2.054(2), Au1-N2=2.613(3), Au1-Cl1=2.2880(7), Au1-Cl2=2.272(7), Au1-Cl3=2.2881(7), N1-Au1-Cl2=177.62(7), N1-Au1-Cl3=89.19(7), Cl1-Au1-Cl3=90.42(3), N1-Au1-Cl1=88.87(7), Cl2-Au1-Cl1=91.42(3), Cl3-Au1-Cl1=175.39(3), N1-Au1-N10=71.8(9). 6E [(di-phenylphen)AuCl3] (18): Au1-N1=2.064(7), Au1-N10=2.704(1), Au1-Cl1=2.297(4), Au1-Cl2=2.267(4), Au1-Cl3=2.288(4), Å N1-Au1-Cl2=173.98(4), N1-Au1-Cl3=90.73(4), Cl1-Au1-Cl3=90.85(16), N1-Au1-Cl1=87.72(3), Cl2-Au1-Cl1=90.73(4), Cl3-Au1-Cl1=178.42(16), N1-Au1-N10=71.80(4).

Gold(III) d⁸ systems generally favor four coordinate square planar geometries, thus the pseudo five coordinate geometry described above represents a relatively unusual coordination environment for gold(III) coordination complexes. Shaw and co-workers summarize a handful of studies from the 1960's and 1970's that report using bipyridine and naphthpyridine ligands to obtain [(L)AuCl₃] complexes [23], and as previously stated, Robinson and co-workers reported in 1975 the use of sterically encumbered ^{di-methyl}phen and bi-quinoline ligands to attain unusual distorted square pyramidal gold(III) complexes [15]. Subsequently, it was not until our report in 2009 that the structural characterization of a pseudo five coordinate gold(III) complexes ossessing alkyl-substituted phen ligands was published in the literature [13]. More recently, ^{methyl}bipy ligands have been used independently by both our research group and Shaw, et al. to prepare [(^{R}bipy)AuCl₃] complexes, and Janzen and co-workers have used both (benzothienyl)pyridine and crown thioether ligands to arrive at structurally similar pseudo five coordinate gold(III) complexes [24]. Given the limited occurrence of this structural motif, compounds 13-16 and 18 significantly expand the overall library of known pseudo five coordinate gold(III) complexes, and to our knowledge compounds 14-16 represent the first structurally characterized distorted square pyramidal gold(III) complexes possessing 2-mono-substituted phen ligands or 6-mono-substituted bipy ligands.

The structural characterization of compounds 13-16 and 18 using X-ray diffraction provides insight about the impact of the alkyl and aryl polypyridyl substituents on the geometrical parameters of the resulting pseudo five coordinate gold(III) complexes. It was originally hypothesized that due to decreased steric interactions between the phen/bipy alkyl substituents and the chloride ligands in the square pyramidal base, the mono-substituted phen and bipy ligands might result in shorter Au—Nₐₓᵢₐₗ interatomic distances compared to the original [(^{di-sec-butyl}phen)AuCl₃] (10) and [(^{di-n-butyl}phen)AuCl₃] (11) complexes. However, the data reported here indicate that the [(^{mono-alkyl}phen)AuCl₃] complexes (14-15) have significantly longer Au—Nₐₓᵢₐₗ interatomic distances compared to the [(^{di-alkyl}phen)AuCl₃] complexes (10-14). Specifically, compounds 14-15 have Au—Nₐₓᵢₐₗ interatomic distances of 2.671(19) Å and 2.642(2) Å, respectively, whereas compounds 10-14 have Au—Nₐₓᵢₐₗ interatomic distances ranging from 2.556(3)-2.612(6) Å (see Table 1). This indicates that the electron donating effects of the di-substituted phen ligands actually enhances the Au—Nₐₓᵢₐₗ interaction, suggesting that the change in the overall electrostatic attraction between the gold(III) center and the axial nitrogen lone pair compensates for the potential increase in repulsion with the dz² electron pair. The [(^{mono-sec-butyl}bipy)AuCl₃] complex (18) has an Au—Nₐₓᵢₐₗ interatomic distance [2.613(3) Å] that is intermediate of the mono-substituted and di-substituted alkyl-phen ligands (see Table 1), though this Au—Nₐₓᵢₐₗ distance is similar to, and perhaps slightly longer than the previously reported [(^{di-methyl}bipy) AuCl₃] complex {Au—Nₐₓᵢₐₗ distance=2.605(3) Å [23] or 2.612(6) Å [14]}. The notion that increased electron donating character of the ligand substituents will enhance the Au—Nₐₓᵢₐₗ interaction is further corroborated by the structural data reported by Shaw and co-workers. A complex possessing the 4,4′-6,6′-tetramethyl-bipy ligand [(^{tetra-methyl}bipy)AuCl₃] had a shorter Au—Nₐₓᵢₐₗ interatomic distance than the analogous [(^{di-methyl}bipy)AuCl₃] complex [Au—Nₐₓᵢₐₗ distances=2.5826(14) Å and 2.605(3) Å, respectively] [23], and the [(^{di-methyl}bipy)AuCl₃] complex has a longer Au—Nₐₓᵢₐₗ interatomic distance than the [(^{di-methyl}phen)AuCl₃] complex (12) [Au—Nₐₓᵢₐₗ distances=2.605(3) Å and 2.58(1) Å, respectively]. The [(^{di-phenyl}phen)AuCl₃] complex (16) has the longest Au—Nₐₓᵢₐₗ interatomic distance among the gold(III) complexes reported here [2.704(12) Å; see Table 1]. Whether this is a steric or electronic effect is difficult to ascertain as the bulky rigid phenyl substituents could certainly create increased steric interaction with the proximal chloride ligands, but the electron density from the phen ligand could also be delocalized into the phenyl substituents, thereby decreasing the electrostatic attraction between the axial nitrogen donor and the gold(III) center. In summary, it is clear that the Au—Nₐₓᵢₐₗ interatomic distance can be tuned by changing the ligand backbone, the nature of the phen/bipy substituent, and/or the number of alky/aryl groups attached to the ligand.

TABLE 1
Bond angles and interatomic distances for the gold-nitrogen interactions in
compounds 10-16, 18. These structural parameters highlight the distorted
square pyramidal geometry around the gold(III) center in this class of pseudo
five coordinate complexes

| | Bond Angle (°) | | Interatomic Distance (Å) | |
|---|---|---|---|---|
| Structure | N—Au—N | Cl—Au—N | Au—Nₐₓᵢₐₗ | Au—N_{equatorial} |
| [(^{di-sec-butyl}phen)AuCl₃] (10) | 71.8(2) | 113.16(18) | 2.612(6) | 2.067(5) |
| [(^{di-n-butyl}phen)AuCl₃]¹⁵ (11) | 73.8(3) | 108.4(2) | 2.597(9) | 2.066(9) |
| [(^{di-methyl}phen)AuCl₃]²³ (12) | 73.2(5) | 111.2(3) | 2.58(1) | 2.09(1) |
| [(^{di-sec-butyl-methyl}phen)AuCl₃] (13) | 73.51(12) | 108.34(8) | 2.556(3) | 2.072(3) |
| [(^{mono-sec-butyl}phen)AuCl₃] (14) | 71.54(6) | 110.66(4) | 2.671(19) | 2.056(17) |
| [(^{mono-n-butyl}phen)AuCl₃] (15) | 72.24(8) | 109.49(6) | 2.642(2) | 2.055(2) |
| [(^{di-phenyl}phen)AuCl₃] (16) | 71.08(4) | 114 08(3) | 2.704(12) | 2.064 (12) |
| [(^{mono-sec-butyl}bipy)AuCl₃] (18) | 71.87(9) | 110.51(6) | 2.613(3) | 2.054(2) |

Another noticeable feature of complexes 10-12 and 16 is that despite the fact distorted square pyramidal complexes have two distinct coordination environments for the nitrogen donor atoms in the solid state, the ¹H NMR spectra reveal that in solution these two sites appear to rapidly exchange. This is evidenced by the observation that the symmetric 2,9-di-substituted ligands lacking the 4-methyl group only have one set of resonances for each proton in the alky substituents and only three sets of aromatic resonances were observed in the ligand backbone. Shaw et al. have previously described this phenomenon and demonstrated that low temperature NMR experiments can be used to confirm that the non-equivalent nitrogen donors can be detected [23]. It is assumed that complexes 10-12 and 16 undergo a similar dynamic exchange of nitrogen donor atoms between the axial and equatorial positions in the solution state, though the pseudo five coordinate geometry is evident in solution for the [^{methyl-di-sec-butyl}phenAuCl₃] complex (13); the overlapping sec-butyl —CH signals in the free ligand (4) are clearly resolved in compound 13 (the overlapping —CH signals at 3.30 ppm in 4 shift to two distinct resonances at 4.38 and 4.66 ppm in 13; see Experimental section).

TABLE 2
X-ray crystal structure refinement data for compounds 13-16, 18.

| | 13 | 14 | 15 | 16 | 18 |
|---|---|---|---|---|---|
| Empirical | C₂₁H₂₆AuCl₃N₂ | C₁₆H₁₆AuCl₃N₂ | C₁₆H₁₆AuCl_{3.5}K_{0.05}N₂ | C₂₄H₁₆AuCl₃N₂ | C₁₄H₁₆AuCl₃N₂ |
| formula | | | | | |
| Formula weight | 609.75 | 546.47 | 542.98 | 635.7 | 515.6 |
| Temperature | 100(2) K | 100(2) K | 100(2) K | 210(2) K | 100(2) K |
| Wavelength | 0.71073 Å | 0.71073 Å | 0.71073 Å | 0.71073 Å | 0.71073 Å |
| Crystal system | Monoclinic | Trigonal | Monoclinic | Monoclinic | Monoclinic |
| Space group | P2(1)/c | R-3 | P 21/n | P2(l)/c (#14) | P 21/n (#14) |
| Unit cell | a = 15.2508(8) Å | a = 19.2996(7) Å | a = 11.8362(4) Å | a = 9.0426(3) Å | a = 12.4047(4) Å |
| dimensions | b = 16.6979(9) Å | b = l9.2996(7) Å | b = 8.7168(3) Å | b = 17.2193(6) Å | b = 8.8762(3) Å |
| | c = 17.3582(9) Å | c = 24.2303(9) Å | c = 16.5670(6) Å | c = 13.6431(4) Å | c = 15.1082(5) Å |
| Volume | 4403.6(4) Å3 | 7816.0(5) Å3 | 1703.0(1) Å3 | 2122.73(12) Å3 | 1622.65(9) Å3 |
| Z | 8 | 18 | 4 | 4 | 4 |
| Density | 1.839 Mg/m3 | 2.090 Mg/m3 | 2.118 Mg/m3 | 1.989 Mg/m3 | 2.111 Mg/m3 |
| (calculated) | | | | | |
| Absorption | 7.054 mm-1 | 8.930 mm-1 | 9.124 mm-1 | 7.323 mm-1 | 9.551 mm-1 |
| coefficient | | | | | |
| F(000) | 2368 | 4674 | 1030 | 1216 | 976 |
| Crystal size | 0.49 × 0.09 × 0.08 | 0.30 × 0.17 × 0.12 | 0.212 × 0.086 × | 0.30 × 0.29 × 0.21 | 0.305 × 0.270 × |
| | mm3 | mm3 | 0.024 mm3 | mm3 | 0.093 mm3 |
| Theta range for | 1.70 to 29.57°. | 1.48 to 30.50°. | 2.035 to 30.507°. | 1.91 to 35.63°. | 1.928 to 30.507°. |
| data collection | | | | | |
| Index ranges | −21 <= h <= 21, | −27 <= h <= 27, | −16 <= h <= 16, | −14 <= h <= 14, | −17 <= h <= 17, |
| | −23 <= k <= 23, | −27 <= k <= 27, | −12 <= k <= 12, | −28 <= k <= 28, | −12 <= k <= 12, |
| | −24 <= l <= 24 | −34 <= l <= 34 | −23 <= l <= 23 | −22 <= l <= 22 | −21 <= l <= 21 |
| Reflections | 96076 | 62889 | 37897 | 151496 | 45188 |
| collected | | | | | |
| Independent | 12353[R(int) = | 5310[R(int) = | 5202[R(int) = | 9796[R(int) = | 4952[R(int) = |
| reflections | 0.0354] | 0.0281] | 0.0396] | 0.0304] | 0.0267] |
| Completeness | 100.00% | 100.00% | 100.00% | 100.00% | 100.00% |
| to theta = | | | | | |
| 30.50° | | | | | |
| Absorption | Semi-empirical | Semi-empirical | Semi-empirical | Semi-empirical | Semi-empirical from |
| correction | from equivalents | from equivalents | from equivalents | from equivalents | equivalents |
| Max. and min. | 0.5988 and 0.1299 | 0.4240 and 0.1734 | n/a | 0.3112 and 0.2174 | n/a |
| transmission | | | | | |
| Refinement | Full-matrix least- | Full-matrix least- | Full-matrix least- | Full-matrix least- | Full-matrix least- |
| method | squares on F2 | squares on F2 | squares on F2 | squares on F2 | squares on F2 |
| Data/restraints/ | 12353/303/608 | 5310/105/252 | 5202/0/218 | 9796/0/271 | 4952/120/222 |
| parameters | | | | | |
| Goodness-of-fit | 1.083 | 1.05 | 1.039 | 1.042 | 1.041 |
| on F2 | | | | | |
| Final R indices | R1 = 0.0340, | R1 = 0.0164, | R1 = 0.0202, | R1 = 0.0173, | R1 = 0.0204, |
| [I > 2 sigma(I)] | wR2 = 0.0799 | wR2 = 0.0532 | wR2 = 0.0410 | wR2 = 0.0385 | wR2 = 0.0488 |
| R indices (all | R1 = 0.0495, | R1 = 0.0183, | R1 = 0.0280, | R1 = 0.0223, | R1 = 0.0231, |
| data) | wR2 = 0.0878 | wR2 = 0.0542 | wR2 = 0.0435 | wR2 = 0.0399 | wR2 = 0.0501 |
| Largest diff. | 3.564 and | 1.310 and | 1.176 and −1.153e · Å−3 | 1.181 and −0.616e · Å−3 | 1.969 and −1.120e · Å−3 |
| peak and hole | −1.545e · Å−3 | −1.452e · Å−3 | | | |

### 3.3 Glutathione Stability and In Vitro Antitumor Activity

It has been shown that resistance to cisplatin treatment by tumor cells is closely correlated to increased levels of intracellular glutathione[25]. Given the fact that gold(III) compounds are often susceptible to reduction the original molecular design strategy for compounds 10 and 11 was centered on the premise that the 2,9-di-substituted phen ligand would impart greater redox stability to the gold(III) center, which in turn would guard this class of metal based drugs against inactivation/reduction by glutathione. It was thought this would occur due to a combination of the steric protection provided by the n-butyl and sec-butyl substituents and the potential enhancement of the ^{R}phen-gold(III) coordinate covalent bonds resulting from the electron donating nature of the alkyl substituents. Indeed, previous studies in our laboratory confirm that the [(^{di-sec-butyl}bipy)AuCl₃] complex (10) has significantly enhanced stability in the presence of reduced glutathione (GSH) compared to a traditional square planar gold(III) complex ion possessing 5,6-dimethyl-1,10-phenanthroline {[(^{5,6-di-methyl}phen)AuCl₂]⁺}. Compound 10 was quite stable in the presence of a GSH buffer solution, as a slow decrease in the LMCT was attributed to the formation of a potential gold(I) species, though neither complete conversion to gold(I) nor any evidence of a gold(0) decomposition product was observed [14]. Conversely, the [(^{5,6-di-methyl}phen)AuCl₂]⁺ square planar complex ion underwent immediate reduction to gold(0) in the presence of GSH, evidenced by the disappearance of the LMCT absorption maximum and the formation of a broad absorption maximum between 550-650 nm [2]. The formation of colloidal gold was observed within five minutes of the GSH addition.

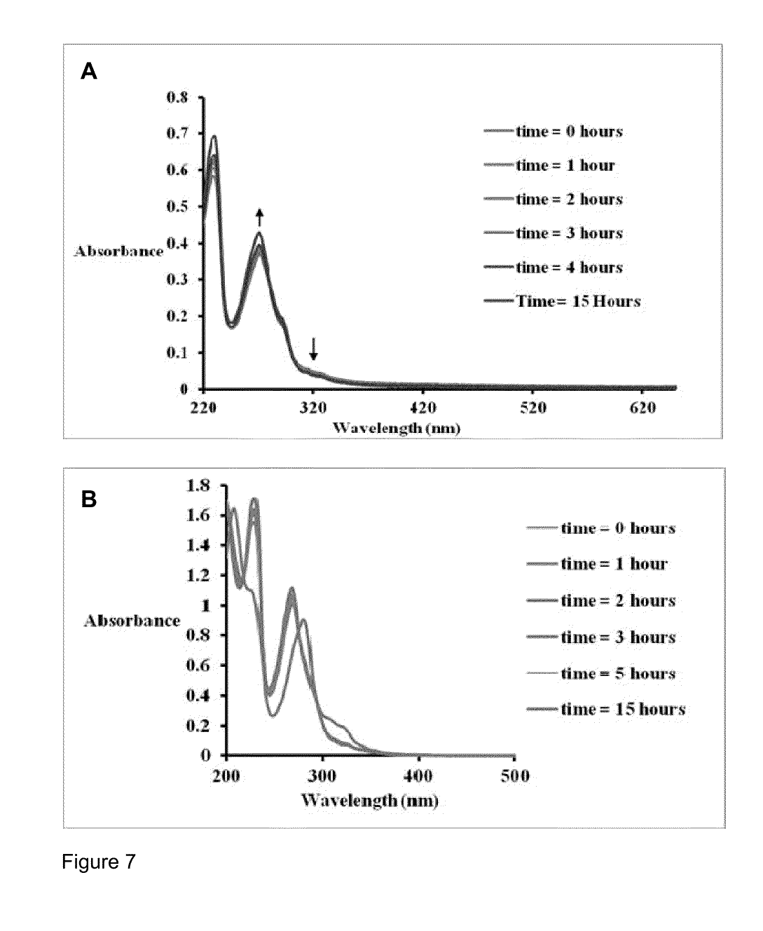

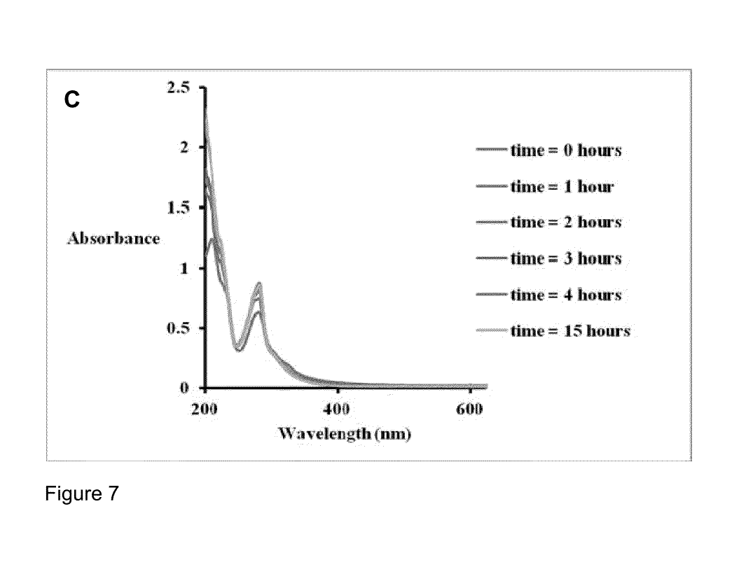

Stability experiments for compounds 11-18 were therefore carried out in order to determine if the resistance to reduction in the presence of GSH is a general property for this class of pseudo five coordinate gold(III) complexes. Compounds 12-14 and 18 exhibited nearly identical stability profiles as compound 10, as a slow and partial decrease in the LMCT band between 300-350 nm and a slow increase in the ligand-centered absorption maximum between 270-300 nm were detected (see FIG. 7). This suggests that partial conversion to a gold(I) thiolate complex likely occurred, though no reduction to colloidal gold(0) was observed. Compound 15 did not experience any decrease in the absorption maxima at 278 nm or 320 nm suggesting no reduction to gold(I) or gold(0) occurred under these reaction conditions. Compounds 11 and 16-17 had slightly different solution behavior, as a slow and partial decrease was observed for both the LMCT between 300-350 nm and the intraligand absorption between 270-300 nm. This is attributed to the formation of insoluble gold(III) hydroxo species, which formed subsequent to the substitution of chloride ligands by aqua ligands. This phenomenon is evidenced not only by the slow decrease in concentration of the original [(^{R}phen)AuCl₃] complexes but by the formation of a colorless precipitate over the course of 15 hours, and has been previously observed with the [(^{di-sec-butyl}phen)AuCl₃] complex (10) in the presence of phosphate buffer [13]. Despite this partial conversion to a [(^{R}phen)Au(Cl₃₋ₓ)(OH)ₓ] complex, compounds 11 and 16-17 still appear to exhibit general redox stability in the presence of GSH. The fact that the formation of colloidal gold(0) was not observed with compounds 10-18 suggests the distorted square pyramidal structural motif is a suitable molecular design choice for limiting the glutathione inactivation of these gold(III) drugs. It is noted that the formation of gold(I) species does not preclude these complexes from being applied as anticancer therapies as it has been previously reported that gold(III) drugs might be activated upon being reduced to gold(I) [26]. Compounds 10-18 therefore appear to possess a potentially ideal redox stability profile, as these pseudo five coordinate complexes are likely to undergo reduction to gold(I) in the reducing intracellular environment of tumor cells yet not likely to be deactivated via reduction to gold(0).

As stated in the introduction, it has been previously reported that even though the [(^{di-sec-butyl}phen)AuCl₃] complex (10) exhibited significant in vitro antitumor activity against a panel of five head-neck and lung tumor cell lines, it was also found that the ^{di-sec-butyl}phen ligand had even more pronounced efficacy than the parent gold(III) complex [14]. Despite the fact the GSH solution study for compound 10 suggests the gold complex likely exhibits suitable stability in the intracellular tumor environment, this result does not eliminate the possibility that the ^{di-sec-butyl}phen ligand is released within the cell and subsequently acts as the active drug. Though other research groups have reported that phen and terpy (2,2′:6,2″-terpyridine) ligands exhibit in vitro antitumor efficacy that is equal to or better than the corresponding gold(III) complexes [27-28], more thorough analyses aiming to determine if the parent ligands are simply released from gold-based drugs in the intracellular tumor environment are often lacking in studies of metallotherapeutic compounds.

In order to gain insight about whether the antitumor activity of the [(^{R}polypyridyl)AuCl₃] class of compounds results from a distinct mechanism of tumor cell death or if these complexes might simply release the active polypyridyl drugs, in vitro SRB assays were carried out for compounds 1-18. All of the ligand and gold(III) complex pairs were tested against the A549 human-derived lung cancer tumor cell line and the SRB growth curves were done in triplicate in order to obtain an average IC₅₀ value for each compound (see Table 3 for IC₅₀ values). All of the polypyridyl ligands (compounds 2-9) exhibited IC₅₀ values that were significantly lower than the cisplatin positive control, though none were as potent as the original ^{di-sec-butyl}phen ligand (1). Interestingly, the activity of the corresponding gold(III) complexes did not change as a function of the ligand activity. Particularly noteworthy are the n-butyl substituted complexes 11 and 15, which both possessed IC₅₀ values significantly lower than their corresponding ^{R}phen ligands; compounds 11 and 15 were approximately 3 and 8 times more active than the free ligands, respectively. These two data points provide strong evidence that the activity of the gold complexes is independent of the polypyridyl ligand since compounds 11 and 15 would not be able to inhibit tumor cell growth significantly more than the free ligands if they were simply releasing the ligands in the intracellular environment. Additionally, it was found that the activity of the [^{methyl-di-sec-butyl}phenAuCl₃] complex (13) was approximately 6 times less active than the corresponding free ligand (4), despite the fact the gold(III) complex demonstrated similar GSH stability to the [^{di-sec-butyl}phenAuCl₃] complex (1). This also suggests the gold complex remains intact and initiates tumor cell death in a ligand-independent fashion, since release of the free ligand from 13 would be expected to result in similar inhibition of tumor cell growth compared to the ^{methyl-di-sec-butyl}phen ligand (4). Finally, it was found that the ^{mono-sec-butyl}bipy ligand (9) and [^{mono-sec-butyl}bipyAuCl₃] complex (18) did not reduce tumor cell growth at any of the concentrations used for compounds 1-8 and 10-17. This indicates that the ligand and gold(III) complex antitumor mechanisms, now presumed to be independent of one another, are both sensitive to structural changes in the polypyridyl ligand backbone.

Compounds 11 and 15 are to date the most potent inhibitors of in vitro tumor cell growth tested in our laboratory, even compared to the potent ^{di-sec-butyl}phen ligand (1). These complexes have IC₅₀ values that are approaching the nanomolar concentration regime and are found to be approximately 12 times lower than cisplatin. Though one must be careful in comparing these results to the in vitro activity of gold complexes reported in other laboratories, we do note that compounds 11 and 15 appear to have antiproliferative effects against in vitro A549 cells that are on par with previously reported gold(III) complexes possessing dithiocarbamate ligands (A549 IC₅₀=0.3-5 μM) [29] and significantly more potent than recently reported gold(I) complexes bearing N-heterocyclic carbenes (A549 IC₅₀=6-100 μM) [30]. Compounds 11 and 15 will therefore be the focus of future drug development.

TABLE 3
IC₅₀ (inhibitory concentration 50%) values for compounds 1-18 and
the positive control cisplatin. SRB colorimetric assays were performed,
and the percentage of cell growth compared to untreated cell cultures
was plotted as a function of drug concentration. Best-fit plots were used
to extrapolate the IC₅₀ values (reported in μM).

| Compound | IC₅₀ (μM) |
|---|---|
| ^{di-sec-butyl}phen (1) | 0.42 ± 0.17 |
| [(^{di-sec-butyl}phen)AuCl₃] (10) | 0.58 ± 0.15 |
| ^{di-n-butyl}phen (2) | 1.08 ± 0.28 |
| [(^{di-n-butyl}phen)AuCl₃] (11) | 0.34 ± 0.06 |
| ^{di-methyl}phen (3) | 1.35 ± 0.09 |
| [(^{di-methyl}phen)AuCl₃] (12) | 1.47 ± 0.21 |
| ^{methyl-di-sec-butyl}phen (4) | 0.70 ± 0.11 |
| [(^{methyl-di-sec-butyl}phen)AuCl₃] | 4.02 ± 0.32 |
| (13) | |
| ^{mono-sec-butyl}phen (5) | 0.62 ± 0.16 |
| [(^{mono-sec-butyl}phen)AuCl_{3]} (14) | 0.77 ± 0.07 |
| ^{mono-n-butyl}phen (6) | 2.92 ± 0.47 |
| [(^{mono-n-butyl}phen)AuCl₃] (15) | 0.37 ± 0.09 |
| ^{di-phenyl}phen (7) | 1.87 ± 0.21 |
| [(^{di-phenyl}phen)AuCl₃] (16) | 1.65 ± 0.13 |
| ^{methyl-di-phenyl}phen (8) | 1.35 ± 0.11 |
| [(^{methyl-di-phenyl}phen)AuCl₃] (17) | 1.37 ± 0.11 |
| ^{mono-sec-butyl}bipy (9) | 100% Cell Growth @ 12 μM |
| [(^{mono-sec-butyl}bipy)AuCl₃] (18) | 100% Cell Growth @ 12 μM |
| cisplatin | 4.67 ± 0.21 |

### 4. CONCLUSION

We have demonstrated that alky- and aryl-substituted phen and alkyl-substituted bipy ligands can be used to conveniently access a broad library of distinctive pseudo five coordinate gold(III) complexes. This class of compounds generally demonstrates suitable redox stability in the presence of reduced glutathione and possesses in vitro antitumor activity against human-derived lung cancer cells that is significantly more pronounced than the clinically used drug cisplatin. Moreover, comparing the antitumor efficacy of the [^{R}polypyridylAuCl₃] complexes to the corresponding free ligands provides evidence that this class of gold(III) compounds very likely has a mechanism of initiating tumor cell death that is independent of the polypyridyl ligand.

### Notes

^{a}X-ray quality crystals of Compound 17 were obtained and a preliminary structure reveals that this complex also possesses the distorted square pyramidal geometry. However, adequate refinement of the structure could not be completed and the crystal structure cannot be included in this report. The elemental analysis and mass spectrometry data do confirm that the [(^{di-phenyl-methyl}phen)AuCl₃] complex was isolated (see Experimental Section).

The following publications relate to Example 3 and are incorporated by reference herein:
[1] L. R. Kelland, in: B. Lippert (Ed.), Verlag Helvetica Chimica Acta, Zurich, Wiley-VCH, Weinheim, Germany, (1999) 497-521.
[2] A. Wein, A. Stockhausen, K. I. Hardcastle, S. Peng, G. Chen, R. Saadein, J. F. Eichler, J. Inorg. Biochem. 105 (5) (2011) 663-668.
[3] R. Sun, Che, C., Coordination Chemistry Reviews 253 (2009) 1682-1691.
[4] L. Messori, F. Abbate, G. Marcon, P. Orioli, M. Fontani, E. Mini, T. Mazzei, S. Carotti, T. O'Connell, P. Zanello, Journal of Medicinal Chemistry 43 (2000) 3541-3548.
[5] M. A. Cinellu, L. Maiore, M. Manassero, A. Casini, M. Arca, H. H. Fiebig, G. Kelter, E. Michelucci, G. Pieraccini, C. Gabbiani, L. Messori, ACS Medicinal Chemistry Letters, 1 (2010) 336-339.
[6] R. M. Snyder, C. K. Mirabelli, S. T. Crooke, Biochem. Pharmacol. 35 (1986) 923.
[7] M. Gielen, E. Tiekink (Eds.), Metallotherapeutic Drugs and Metal-Based Diagnostic Agents: The Use of Metals in Medicine, Wiley and Sons, West Sussex, England, 2005.
[8] L. Messori, P. Orioli, C. Tempi, G. Marcon, Biochem. And Biophys. Research Comm. 281 (2001) 352.
[9] M. P. Rigobello, L. Messori, G. Marcon, M. A. Cinellu, M. Bragadin, A. Folda, G. Scutari, A. Bindoli, J. Inorg. Biochem. 98 (2004) 1634-1641.
[10] F. Mendes, M. Groessl, A. A. Nazarov, Y. O. Tsybin, G. Sava, I. Santos, P. J. Dyson, A. Casini, J. Med. Chem. 54 (2011) 2196-2206.
[11] V. Milacic, Q. P. Dou, Coord. Chem. Rev. 253 (2009) 1649-1660.
[12] K. Fritz-Wolfl, S. Urig, K. Becker, J. Mol. Biol. 370 (2007) 116.
[13] Z. Hudson, C. Sanghvi, M. Rhine, J. J. Ng, S. D. Bunge, K. I. Hardcastle, R. Saadein, C. E. MacBeth, J. F. Eichler, Dalton Transactions (2009) 7473-7480.
[14] C. D. Sanghvi, P. M. Olsen, C. Elix, S. B. Peng, D. Wang, Z. G. Chen, D. M. Shin, K. I. Hardcastle, C. E. Macbeth, J. F. Eichler, J Inorg Biochem. 128 (2013) 68-76.
[15] W. T. Robinson, E. Sinn, J. Chem. Soc. Dalton Trans. (1975) 726.
[16] APEX 2, version 2012.2.2, Bruker (2012), Bruker AXS Inc., Madison, Wis., USA.
[17] SAINT, version V8.18C, Bruker (2011), Bruker AXS Inc., Madison, Wis., USA.
[18] SADABS, version 2008/1, Bruker (2008), Bruker AXS Inc., Madison, Wis., USA.
[19] SHELXTL, version 2008/4, Bruker (2008), Bruker AXS Inc., Madison, Wis., USA.
[20] P. Skehan, R. Storeng, D. Scudiero, A. Monks, J. McMahon, D. Vistica, J. T. Warren, H. Bokesch, S. Kenney, M. R. Boyd, J Natl Cancer Inst. 82 (1990) 1107-1112.
[21] A. J. Pallenberg, K. S. Koenig, D. M. Barnhart, Inorg. Chem. 34 (1995) 2833.
[22] S. Jakobsen, M. Tilset, Tetrahedron Letters, 52 (2011) 3072-3074.
[23] A. P. Shaw, M. K. Ghosh, K. W. Tornroos, D. S. Wragg, M. Tilset, 0. Swang, R. H. Heyn, S. Jakobsen, Organometallics, 31 (2012) 7093-7100.
[24] D. E. Janzen, S. R. Doherty, D. G. VanDerveer, L. M. Hinkle, D. A. Benefield, H. M. Vashi, G. J. Grant, J. Organomet. Chem. 755 (2014) 47-57.
[25] A. K. Goodwin, A. Meister, P. J. O'Dwyer, C. S. Huang, T. C. Hamilton, M. E. Anderson, Proc. Natl. Acad. Sci. 89 (1992) 3070-3074.
[26] M. A. Cinellu, L. Maiore, M. Manassero, A. Casini, M. Arca, H. H. Fiebig, G. Kelter, E. Michelucci, G. Pieraccini, C. Gabbiani, L. Messori, Med. Chem. Lett. (2010) 336-339.
[27] L. Messori, F. Abbate, G. Marcon, P. Orioli, M. Fontani, E. Mini, T. Mazzei, S. Carotti, T. O'Connell, P. Zanello, J. Med. Chem. 43 (2000) 3541-3548.
[28] P. Shi, Q. Jiang, Y. Zhao, Y. Zhang, J. Lin, L. Lin, J. Ding, Z. Guo, J. Biol. Inorg. Chem. 11 (2006) 745-752.
[29] L. Ronconi, L. Giovagnini, C. Marzano, F. Betti'o, R. Graziani, G. Pilloni, D. Fregona, Inorg. Chem. 44 (2005) 1867-1881.
[30] L. Messori, L. Marchetti, L. Massai, F. Scaletti, A. Guerri, I. Landini, S. Nobili, G. Perrone, G. Enrico, P. Leoni, M. Pasquali, C. Gabbia, Inorg. Chem. 53 (2014) 2396-2403.

### Example 4

The in vitro antitumor activity of 2,9-di-sec-butyl-1,10-phenanthroline, (2,9-di-sec-butyl-1,10-phenanthroline)AuCl₃, and (2-mono-n-butyl-phenanthroline)AuCl₃ was determined on GL-26 murine glioma cells.

Cell Lines:

The murine (C57BL/6) glioma cell line, GL-26, which is highly tumorigenic in the C57BL/6 mice, was used. GL-26 cells were cultured in DMEM/F12 supplemented with 10% FCS, 1% penicillin/streptomycin, 1% L-glutamine and 1% non-essential amino acids. Human foreskin fibroblasts (HFFs) were cultured in DMEM/F12 supplemented with 10% FCS and 1% penicillin/streptomycin. Primary murine astrocytes were purified from C57BL/6 neonate brains and cultured in DMEM/F12 supplemented with 10% FCS, 1% non-essential amino acids, 1% L-glutamine, 50 IU/ml penicillin, 50 mg/ml streptomycin and 10 mM Hepes buffer.

Growth Assay:

The sulforhodamine B (SRB) cytotoxicity assays were adapted from Skehan et al (21). Briefly, either HFF, primary astrocytes or GL-26 cells were plated in 96-well plates at a density of 4,000 cells/well in a volume of 1004 overnight at 37° C. and 5% CO₂. DMSO stock solutions of SBP, MSBP or TMZ were used at a concentration range of 0.1-25 μM for 48 hr before the supernatant was discarded and the cells were fixed for 1 hr with 10% cold trichloroacetic acid (100 μL per well). Cells used in recovery assay received fresh media for 48 hrs following the 48 hr drug incubation. The plate was then washed 5 times with de-ionized water, air dried, and stained with 0.4% SRB for 10 min (50 μL per well). After washing 5 times in 1% acetic acid and air-drying, bound SRB was dissolved in 10 mM unbuffered Tris base (pH 10.5; 100 μL per well). Bound SRB was then read by absorbance at 492 nm on a SpectraMax plate reader (Molecular Devices). The percent survival was then calculated based upon the absorbance values relative to control wells (0 μM SBP in 0.1% DMSO).

### Results

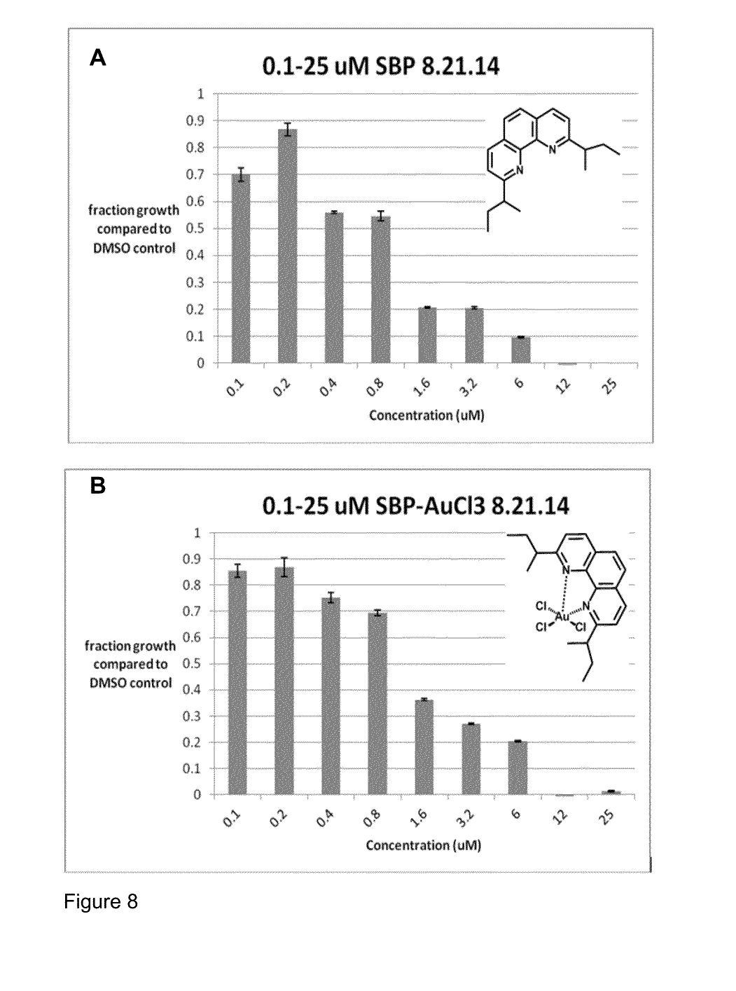

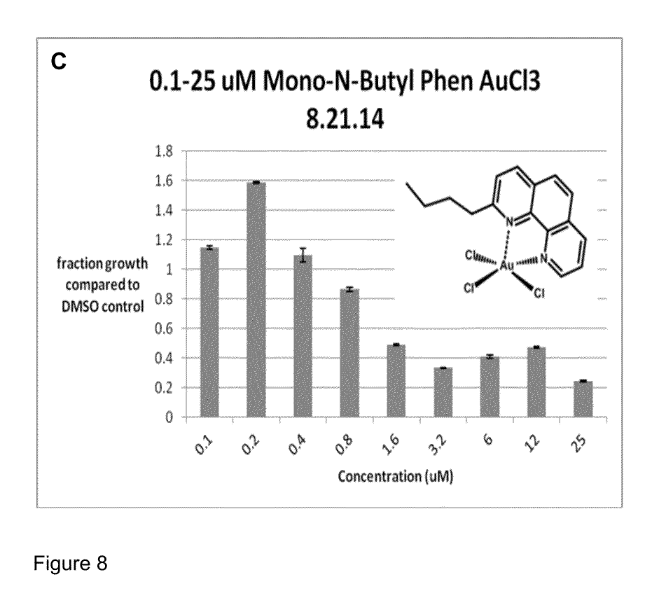

As shown in FIG. 8, the gold complexes have antitumor activity that is similar to SBP alone. The in vitro antitumor activity of SBP (FIG. 8A) was compared to the [(^{di-sec-butyl}phen)AuCl₃] (FIG. 8B) and [(^{mono-n-butyl}phen)AuCl₃] (FIG. 8C) complexes. All three compounds were found to inhibit GL-26 by 50% between 0.8 and 1.6 μM.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of inhibiting glioma cell growth, comprising
exposing glioma cells to at least one antitumor compound in an amount effective to inhibit growth of the glioma cells,
wherein the antitumor compound is selected from the group consisting of: 2,9-di-sec-butyl-1,10-phenanthroline; (2,9-di-sec-butyl-1,10-phenanthroline)AuCl₃; (2-mono-n-butyl-phenanthroline)AuCl₃; and a combination thereof.

2. The method of claim 1, wherein the glioma cells are glioblastoma cells.

3. The method of claim 1, further comprising exposing the cells to an anticancer agent.

4. The method of claim 3, wherein the anticancer agent is a platinum-based compound.

5. The method of claim 4, wherein the platinum-based compound is cisplatin.

6. A method of treating a glioma in a subject in need of such treatment, comprising
administering at least one antitumor compound to the subject in an amount effective to treat the tumor,
wherein the antitumor compound is selected from the group consisting of: 2,9-di-sec-butyl-1,10-phenanthroline; (2,9-di-sec-butyl-1,10-phenanthroline)AuCl₃; (2-mono-n-butyl-phenanthroline)AuCl₃; and a combination thereof.

7. The method of claim 6, wherein the glioma is a glioblastoma.

8. The method of claim 6, further comprising administering an anticancer agent to the subject.

9. The method of claim 8, wherein the anticancer agent is a platinum-based compound.

10. The method of claim 9, wherein the platinum-based compound is cisplatin.

11. The method of claim 6, further comprising administering an anticancer treatment to the subject.

12. The method of claim 11, wherein the anticancer treatment is surgery, chemotherapy, radiotherapy or immunotherapy.

13. A method of inhibiting cancer cell growth, comprising
exposing cancer cells to at least one antitumor compound in an amount effective to inhibit growth of the cancer cells,
wherein the cancer cells are lung cancer or glioma cancer cells, and the antitumor compound is selected from the group consisting of:
2,9-di-n-butyl-1,10-phenanthroline;
2,9-di-sec-butyl-4-methyl-1,10-phenanthroline;
2-sec-butyl-1,10-phenanthroline;
2-mono-n-butyl-phenanthroline;
2,9-di-phenyl-1,10-phenanthroline;
2,9-di-phenyl-4-methyl-1,10-phenanthroline;
2-mono-sec-butyl-2,2′-bipyridine;
(2,9-di-n-butyl-1,10-phenanthroline)AuCl₃;
(2,9-di-methyl-1,10-phenanthroline)AuCl₃;
(2,9-di-sec-butyl-4-methyl-1,10-phenanthroline)AuCl₃;
(2-sec-butyl-1,10-phenanthroline)AuCl₃;
(2-mono-n-butyl-phenanthroline)AuCl₃;
(2,9-di-phenyl-1,10-phenanthroline)AuCl₃;
(2,9-di-phenyl-4-methtyl-1,10-phenanthroline)AuCl₃;
2-mono-sec-butyl-2,2′-bipyridine)AuCl₃; and
a combination thereof.

14. The method of claim 13, wherein the cancer cells are lung cancer cells.

15. The method of claim 13, wherein the cancer cells are glioma cells.

16. The method of claim 13, wherein the compound is (2,9-di-n-butyl-1,10-phenanthroline)AuCl₃ or (2-mono-n-butyl-phenanthroline)AuCl₃.

17. A method of treating cancer in a subject in need of such treatment, comprising
administering at least one antitumor compound to the subject in an amount effective to treat the cancer,
wherein the cancer is lung cancer or glioma cancer, and the antitumor compound is selected from the group consisting of:
2,9-di-n-butyl-1,10-phenanthroline;
2,9-di-sec-butyl-4-methyl-1,10-phenanthroline;
2-sec-butyl-1,10-phenanthroline;
2-mono-n-butyl-phenanthroline;
2,9-di-phenyl-1,10-phenanthroline;
2,9-di-phenyl-4-methyl-1,10-phenanthroline;
2-mono-sec-butyl-2,2′-bipyridine;
(2,9-di-n-butyl-1,10-phenanthroline)AuCl₃;
(2,9-di-methyl-1,10-phenanthroline)AuCl₃;
(2,9-di-sec-butyl-4-methyl-1,10-phenanthroline)AuCl₃;
(2-sec-butyl-1,10-phenanthroline)AuCl₃;
(2-mono-n-butyl-phenanthroline)AuCl₃;
(2,9-di-phenyl-1,10-phenanthroline)AuCl₃;
(2,9-di-phenyl-4-methtyl-1,10-phenanthroline)AuCl₃;
2-mono-sec-butyl-2,2′-bipyridine)AuCl₃; and
a combination thereof.

18. The method of claim 17, wherein the cancer is lung cancer.

19. The method of claim 17, wherein the cancer is a glioma tumor.

20. The method of claim 17, wherein the compound is (2,9-di-n-butyl-1,10-phenanthroline)AuCl₃ or (2-mono-n-butyl-phenanthroline)AuCl₃.

21. A compound selected from the group consisting of:
2,9-di-sec-butyl-4-methyl-1,10-phenanthroline;
2-sec-butyl-1,10-phenanthroline;
2-mono-n-butyl-phenanthroline;
2,9-di-phenyl-1,10-phenanthroline;
2,9-di-phenyl-4-methyl-1,10-phenanthroline;
2-mono-sec-butyl-2,2′-bipyridine;
(2,9-di-sec-butyl-4-methyl-1,10-phenanthroline)AuCl₃;
(2-sec-butyl-1,10-phenanthroline)AuCl₃;
(2-mono-n-butyl-phenanthroline)AuCl₃;
(2,9-di-phenyl-1,10-phenanthroline)AuCl₃;
(2,9-di-phenyl-4-methtyl-1,10-phenanthroline)AuCl₃; and
(2-mono-sec-butyl-2,2′-bipyridine)AuCl₃.

22. A pharmaceutical composition comprising one or a combination of the compounds of claim 21, and a pharmaceutically acceptable carrier.
